(19) United States
(12) Patent Application Publication
(10) Pub. No.: US 2018/0280372 A1
(43) Pub. Date: Oct. 4, 2018

(54) COMPOSITIONS AND METHODS FOR INHIBITING AUTOPHAGY AND CONTRACEPTION

(71) Applicants: Kelle Moley, St. Louis, MO (US); Arin Kettle Oestreich, St. Louis, MO (US)

(72) Inventors: Kelle Moley, St. Louis, MO (US); Arin Kettle Oestreich, St. Louis, MO (US)

(73) Assignee: Washington University, St. Louis, MO (US)

(21) Appl. No.: 15/820,886

(22) Filed: Nov. 22, 2017

Related U.S. Application Data
(60) Provisional application No. 62/425,428, filed on Nov. 22, 2016.

(51) Int. Cl. A61K 31/451; A61K 31/4045; A61K 31/167; A61K 9/00; A61P 15/18

(52) U.S. Cl. CPC A61K 31/451; A61K 31/4045; A61P 15/18; A61K 9/0039; A61K 31/167

(57) ABSTRACT

The present disclosure provides methods and compositions comprising a autophagy inhibitors. Also provided herein are autophagy inhibitors for use as a contraceptive device. Also provided are autophagy inhibitors delivered by an intrauterine delivery system (IUS) to prevent pregnancy or provide contraception. Also provided herein are newly identified compositions for use as autophagy inhibitors.

### CROSS-REFERENCE TO RELATED APPLICATIONS

This application claims priority from U.S. Provisional Application Ser. No. 62/425,428 filed on 22 Nov. 2016, which is incorporated herein by reference in its entirety.

### STATEMENT REGARDING FEDERALLY SPONSORED RESEARCH OR DEVELOPMENT

This invention was made with government support under grant number HD065435 awarded by National Institutes of Health. The government has certain rights in the invention.

### MATERIAL INCORPORATED-BY-REFERENCE

Not applicable.

### FIELD OF THE INVENTION

The present disclosure generally relates to compositions and methods for contraception or pregnancy prevention. More specifically, the compositions can comprise an autophagy inhibitor (e.g., a ULK1 inhibitor) incorporated into a contraceptive device. The autophagy or ULK1 inhibitor can be delivered by an intrauterine delivery system (IUS) to prevent conception.

### BACKGROUND OF THE INVENTION

All reproductive aged women spend a significant portion of their lives at risk of an unintended pregnancy. Unintended pregnancies worldwide are a serious public health concern because of increased risk of adverse social, economic, and physical health outcomes. Encouraging the use of effective and acceptable contraception is critical to decrease these rates. Long-acting reversible contraceptive methods (LARC), including contraceptive implants and intrauterine contraception (copper-releasing and levonorgestrel-releasing devices/systems), are the most effective reversible contraceptive methods. LARCs are known to be highly effective in preventing pregnancy, and the American College of Obstetricians and Gynecologists has recommended the IUD as first-line contraceptive. Despite their popularity, many women discontinue their use due to abnormal bleeding as seen with both the levonorgestrel and copper containing IUD. The struggle is finding a non-hormonal common pathways within the reproductive tract that if targeted would attack both ovulation and implantation. Once discovered, an ideal compound could be designed and delivered via an intrauterine device.

### SUMMARY OF THE INVENTION

Among the various aspects of the present disclosure is the provision of compositions and methods for inhibiting autophagy and methods of contraception or pregnancy prevention.

Briefly, therefore, the present disclosure is directed to non-hormonal methods and compositions for contraception or pregnancy prevention.

The present teachings include methods of contraception comprising administration of a therapeutically effective amount of a composition comprising an autophagy inhibiting agent in a subject at risk for becoming pregnant.

An aspect of the present disclosure provides for a method of contraception comprising administration of a therapeutically effective amount of a composition comprising an autophagy inhibiting agent in a subject at risk for becoming pregnant.

In some embodiments, the autophagy inhibiting agent disrupts uterine receptivity, ovulation, or decidualization.

In some embodiments, the therapeutically effective amount of the autophagy inhibiting agent inhibits autophagy; halts, impairs, or inhibits decidualization; inhibits or prevents pregnancy; inhibit or prevent conception; disrupts ovulation; inhibits or prevents ovulation; reduces fertility or fecundity; reduces corpora lutea; or disrupts uterine receptivity.

In some embodiments, the subject is a fertile female with a uterus.

In some embodiments, the composition does not comprise a synthetic hormone.

In some embodiments, the subject has or is suspected of having a condition in which hormone exposure is contraindicated.

In some embodiments, the composition comprises one or more autophagy inhibiting agents selected from the group consisting of: niclosamide, Zafirlukast, and L(−)-vesamicol hydrochloride.

In some embodiments, the composition comprises one or more autophagy inhibiting agents selected from the group consisting of: SBI-0206965, niclosamide, Zafirlukast, L(−)-vesamicol, MRT68921, and LYN-1604.

Another aspect of the present disclosure provides for a method of contraception comprising administering intrauterinely a composition comprising an autophagy inhibitor.

In some embodiments, the autophagy inhibitor is selected from the group consisting of: niclosamide, Zafirlukast, and L(−)-vesamicol hydrochloride.

In some embodiments, the autophagy inhibitor is selected from the group consisting of: SBI-0206965, niclosamide, Zafirlukast, L(−)-vesamicol, MRT68921, and LYN-1604.

In some embodiments, the composition is formulated as an intrauterine system (IUS) or intrauterine device (IUD).

Another aspect of the present disclosure provides for a delivery system comprising a body construction suitable for use in an intrauterine system comprising at least one pharmaceutical composition comprising an autophagy inhibiting agent.

In some embodiments, the body construction comprises at least one pharmaceutical composition; or the body construction comprises a biocompatible polymer.

In some embodiments, the body construction comprises a capsule; the capsule has at least a first end and a second end; the capsule comprises the at least one pharmaceutical composition comprising a pharmaceutically active agent; the body construction has at least two locking parts, each locking part having at least a first end and a second end; the first end of each locking part has a surface adapted to face and cover one of the at least first and second ends of the capsule; the diameter of at least one of the locking parts varying along its length between the first end and the second end; the capsule is mounted between the at least two locking parts; or the locking parts have a truncated cone shape.

In some embodiments, the body construction comprises two or more capsules containing a pharmaceutical composition.

In some embodiments, the delivery system is an intrauterine system (IUS) or intrauterine device (IUD).

Another aspect of the present disclosure provides for a method of reducing autophagy in a subject comprising administering a pharmaceutical composition comprising an autophagy inhibiting agent selected from the following: niclosamide, Zafirlukast, and L(−)-vesamicol hydrochloride to a subject in need thereof.

In some embodiments, the subject is at risk for becoming pregnant.

In some embodiments, the subject has a disease disorder or condition treatable with an autophagy inhibiting agent selected from the group consisting of metabolic conditions, obesity, diabetes, or cancer.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a series of images showing Endometrial Stromal Cell (ESC) decidualization.

FIG. 2 is a series of images showing Cellular Changes during Decidualization (Favaro et al. (2015) The guide to investigation of mouse pregnancy Chapter 11: “Decidualization and endometrial extracellular matrix remodeling”).

FIG. 3 is an illustration showing autophagy in endometrial stromal cells is required during the periovulatory period in order to break down macromolecules and organelles for cell energy (adapted from Saito and Nakashima 2014 “A review of the mechanism for poor placentation in early-onset preeclampsia: the role of autophagy in trophoblast invasion and vascular remodeling”).

FIG. 4 is an illustration showing autophagy flux assay.

FIG. 5A-FIG. 5B is a western blot and a bar graph showing Autophagy is upregulated during decidualization of human ESCs but impaired in high fat conditions. (FIG. 5A) Western blot of LC3b-II and GAPDH (loading control) in control (Con) and decidualized (Dec) human ESCs in the presence or absence of PA and in the presence or absence of Bafilomycin A1 (BafA1). (FIG. 5B) Quantification of LC3b-II level relative to GAPDH in BafA1-treated cells (n=3-7 treatment wells per condition). Values are expressed as mean±SE. *P<0.001, as analyzed by analysis of variance with the Bonferroni correction for multiple comparisons. This experiment was performed in triplicate.

FIG. 6 illustrates the experimental design of the study showing diet-induced obesity impairs endometrial stromal cell decidualization, showing a potential role for impaired autophagy (inventor publication, Rhee et al., Human Reproduction, Vol. 31, No. 6 pp. 1315-1326, 2016).

FIG. 7A-FIG. 7B is a series of images and bar graphs showing a high fat diet impaired artificial decidualization in mice and human endometrial stromal cells (ESCs) (Rhee et al., 2016).

FIG. 8A-FIG. 8B is a series of bar graphs showing a high fat diet impairs artificial decidualization in mice and human endometrial stromal cells (ESCs) (Rhee et al., 2016).

FIG. 9A-FIG. 9B is a Western Blot and a bar graph showing autophagy is up-regulated during murine decidualization but impaired with high fat diet (Rhee et al., 2016).

FIG. 10 is a series of images and a bar graph showing decidualization is impaired in mice with decreased autophagy.

FIG. 11 is an illustration showing the pharmacologic inhibition of autophagy.

FIG. 12 is a series of bar graphs showing the pharmacologic inhibition of autophagy inhibits decidualization in immortalized human ESCs.

FIG. 14 is a series of bar graphs showing during in vitro decidualization the decidualization markers PRL and IGFBP1 increase in immortalized human ESCs.

FIG. 15 is a series of images showing autophagosomes are larger in decidualized immortalized human ESCs.

FIG. 16 is a series of images and a bar graph showing autophagy is also increased in ESCs cultured from LC3-GFP mice.

FIG. 17 is a bar graph of number of pups for HM, HET, and WT mice (N=6).

FIG. 18 is a bar graph showing ATP levels decrease during decidualization but not under high fat conditions.

FIG. 19 is a series of images, bar graphs, and Western blots showing autophagy increases during decidualization of immortalized human (hESC-t).

FIG. 20 is a series of images and bar graphs showing autophagosomes of decidualizing hESC-t cells contain more cellular cargo.

FIG. 21A-FIG. 21G is a series of images, bar graphs, and Western blots showing decidualization is impaired in Atg16L1 mice with impaired autophagy.

FIG. 22 is a series of images, bar graphs, and whisker plots showing uterine specific knock out of Atg16L1 impairs artificial decidualization and fecundity.

FIG. 23 is an illustration of a pharmacological model of phagophore initiation.

FIG. 24 is a series of Western blots and a bar graph showing Ulk inhibition decreases autophagy in hESCs-ts.

FIG. 25 is a series of images and a bar graphs showing pharmacologic inhibition of autophagy in hESCs-t cells prevents morphological cellular changes indicative of decidualization.

FIG. 26 is a series of images showing Ulk inhibition does not induce apoptosis in hESC-t cells (TUNNEL ASSAY).

FIG. 27 is a series of images and a bar graph showing pharmacologic inhibition of autophagy in primary hESCs-cells prevents morphological cellular changes indicative of decidualization.

FIG. 28 is an illustration showing the superovulation experimental design.

FIG. 29 is a series of images and a bar graph showing an ovulation defect when treated with the autophagy inhibitor.

FIG. 30 is a series of histology images and a bar graph showing corpora lutea are reduced 3 days following autophagy inhibition suggesting impaired ovulation.

FIG. 31 is a series bar graphs showing autophagy inhibition decreases cellular metabolites in the oocyte.

FIG. 32 is a series of images showing other identified inhibitors from a C. elegans drug screen.

FIG. 33 is a chart showing where Zafirlukast blocks the receptor for leukotriene C4, D4, and E4.

FIG. 34 is a series of images and a bar graph showing decidualization is impaired in hESC-t cells treated with Zafirlukast.

FIG. 35 is a series of images and a bar graph showing decidualization is impaired in primary hESC cells treated with Zafirlukast.

### DETAILED DESCRIPTION OF THE INVENTION

The present disclosure is based, at least in part, on the discovery that autophagy is a pathway that can be targeted for contraception or to prevent pregnancy. As shown herein, the present disclosure determined that autophagy is a normal and required process for proper endometrial decidualization and ovulation. Furthermore, disruption of this process can provide contraception or prevent pregnancy.

Autophagy is a cell-protective and degradative process that recycles damaged and long-lived cellular components in response to an acute need for energy. Autophagy in endometrial stromal cells is required during the periovulatory period in order to break down macromolecules and organelles for cell energy.

As described herein, the present disclosure shows that in obese mice, autophagy is halted, decidualization does not occur, and implantation is severely impaired. Similarly, in autophagy-deficient mice (ATG16L1 hypomorphs) litter size is smaller and fertility is impaired. Disturbances are also seen in the regression of the corpus luteum and subsequent ovulation in both models as well.

As described herein, the present disclosure shows that an inhibitor of Ulk1, a serine/threonine protein kinase (presently thought to be required for the initial stages of autophagy) can impair decidualization and ovulation and thus impair fertility. This compound may be delivered by intrauterine pellets and ultimately by an IUS, in order to evaluate the efficacy of this novel target for contraception. The hypothesis is that the normal flux of the autophagic pathway in reproductive tissues is critical for successful pregnancy and thus targeting autophagy as a feasible contraceptive target. A ULK1 inhibitor can be formulated into a LARC IUS as an efficient delivery system. As such, a composition comprising a ULK1 inhibitor can be successful in preventing pregnancy.

The compositions and methods provided herein can be used to inhibit or prevent decidualization. Decidualization is a process that results in significant changes to cells of the endometrium in preparation for, and during, pregnancy.

Autophagy Inhibiting Agent

As described herein, a pharmaceutically active agent can comprise an autophagy inhibiting agent. An autophagy inhibiting agent can be any agent that inhibits autophagy. The autophagy inhibiting agent can be used to inhibit pregnancy or conception, provide contraception, or reduce fertility. The autophagy inhibiting agent can reduce autophagic activity in a subject.

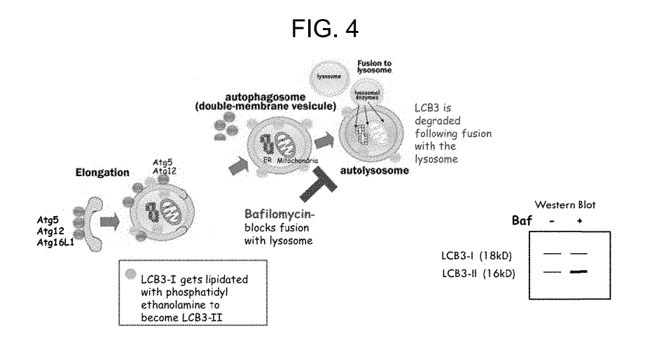

Methods of measuring autophagic activity are well known in the art (see e.g., autophagy flux assay in FIG. 4). Autophagy inhibition agents can be any agent that is capable of inhibiting autophagy. For example, an autophagy inhibitor can be used for preventing pregnancy or providing contraception. As an example, an autophagy inhibiting agent discovered herein can be: niclosamide (PubChemID 4477), Zafirlukast (PubChemID 5717), or L(−)-vesamicol hydrochloride (PubChemID 659840). As another example, an autophagy inhibition agent can be a ULK inhibitor (e.g., SBI-206965).

Other autophagy inhibiting agents that can be used can include known autophagy inhibitors, such as those listed below:

| Product | | | |
|---|---|---|---|
| Name | Targets | Information | Structure |
| ABT-737 | Autophagy, Bcl-2 | ABT-737 is a BH3 mimetic inhibitor of Bcl- xL, Bcl-2 and Bcl-w with EC50 of 78.7 nM, 30.3 nM and 197.8 nM, respectively; no inhibition observed against Mcl-1, Bcl-B or Bfl-1. Phase 2. | 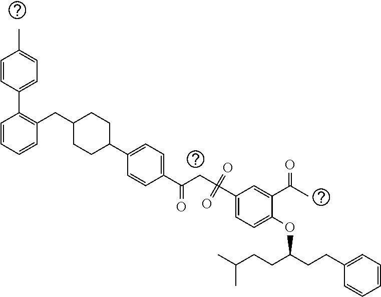 |
| FG-4592 | HIF | FG-4592 is an HIF α prolyl hydroxylase inhibitor, stabilizes HIF-2 and induces EPO production. Phase 2/3. | 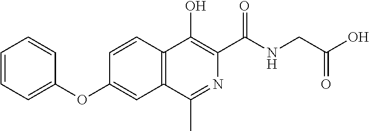 [SMILES: Cc1nc(C(=O)NCC(=O)O)c(O)c2ccc(Oc3ccccc3)cc12] |
| BEZ235 (NVP- BEZ235, Dactolisib) | PI3K,ATM/ ATR, mTOR | BEZ235 (NVP-BEZ235) is a dual ATP- competitive PI3K and mTOR inhibitor for p110α/γ/δ/β and mTOR(p70S6K) with IC50 of 4 nM/5 nM/7 nM/75 nM/6 nM, respectively. Inhibits ATR with IC50 of 21 nM; shown to be poor inhibitory to Akt and PDK1. Phase 1/2. | 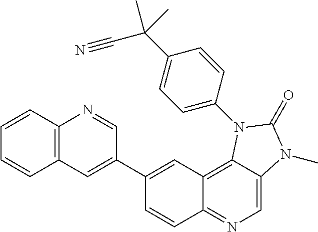 [SMILES: Cn1c(=O)n(-c2ccc(C(C)(C)C#N)cc2)c2c3cc(-c4cnc5ccccc5c4)ccc3ncc21] |
| Bortezomib (PS-341) | Proteasome | Bortezomib (PS-341) is a potent 20S proteasome inhibitor with Ki of 0.6 nM. | 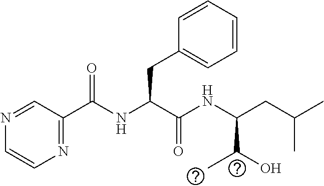 [SMILES: CC(C)C[C@H](NC(=O)[C@H](Cc1ccccc1)NC(=O)c1cnccn1)C(C)O] |
| Ridaforolimus (Deforolimus MK-8669) | mTOR | Ridaforolimus (Deforolimus) is a selective mTOR inhibitor with IC50 of 0.2 nM; while not classified as a prodrug, mTOR inhibition and FKBP12 binding is similar to rapamycin. | 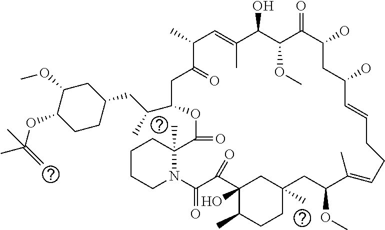 |
| Erlotinib HCl (OSI- 744) | Autophagy, EGFR | Erlotinib HCl (OSI-744) is an EGFR inhibitor with IC50 of 2 nM, >1000-fold more sensitive for EGFR than human c-Src or v- Abl. Phase 3. | 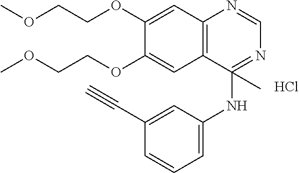 |
| PI-103 | PI3K, Autophagy, DNA- PK, mTOR | PI-103 is a multi-targeted PI3K inhibitor for p110α/β/δ/γ with IC50 of 2 nM/3 nM/3 nM/15 nM, less potent to mTOR/DNA-PK with IC50 of 30 nM/23 nM. | 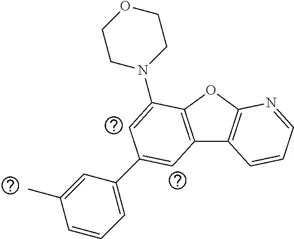 [SMILES: Cc1cccc(-c2cc(N3CCOCC3)c3oc4ncccc4c3c2)c1] |
| Rapamycin (Sirolimus) | Autophagy, mTOR | Rapamycin (Sirolimus, AY-22989, WY- 090217) is a specific mTOR inhibitor with IC50 of ~0.1 nM. | 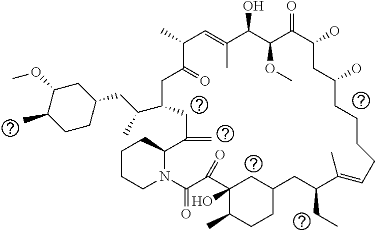 [SMILES: C=C1C[C@H]([C@H](C)C[C@@H]2CC[C@@H](C)[C@H](OC)C2)CC(=O)[C@H](C)/C=C(\C)[C@@H](O)[C@H](OC)C(=O)[C@H](O)C[C@H](O)CCCC/C=C(\C)[C@@H](CC)CC2CC[C@@H](C)[C@@](O)(C2)C(=O)C(=O)N2CCCC[C@@H]12] |
| Temsirolimus (CCI- 779, NSC 683864) | mTOR | Temsirolimus (CCI-779) is a specific mTOR inhibitor with IC50 of 1.76 μM. | 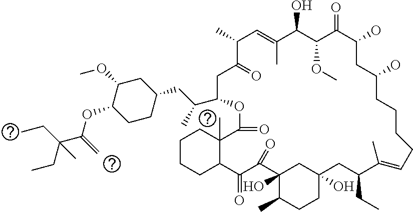 [SMILES: C=C(O[C@H]1CC[C@@H](C[C@@H](C)[C@@H]2CC(=O)[C@H](C)/C=C(\C)[C@@H](O)[C@@H](OC)C(=O)[C@H](O)C[C@H](O)CCCC/C=C(\C)[C@@H](CC)C[C@@]3(O)CC[C@@H](C)[C@@](O)(C3)C(=O)C(=O)C3CCCCC3(C)C(=O)O2)C[C@H]1OC)C(C)(CC)CC] |
| Trichostatin A (TSA) | HDAC | Trichostatin A (TSA) is an HDAC inhibitor with IC50 of ~1.8 nM - HDAC8 is the only known member of the HDAC-family that is not affected by TSA. Phase 3. | 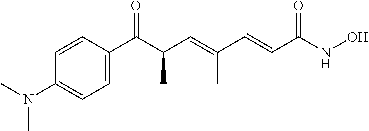 [SMILES: CC(/C=C/C(=O)NO)=C\[C@@H](C)C(=O)c1ccc(N(C)C)cc1] |
| Vorinostat (SAHA, MK0683) | Autophagy, HDAC | Vorinostat (suberoylanilide hydroxamic acid, SAHA) is an HDAC inhibitor with IC50 of ~10 nM. | 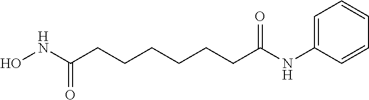 [SMILES: O=C(CCCCCCC(=O)Nc1ccccc1)NO] |
| VX-680 (Tozasertib, MK-0457) | Aurora Kinase | VX-680 (Tozasertib, MK-0457) is a pan- Aurora inhibitor, mostly against Aurora A with Kiapp of 0.6 nM, less potent towards Aurora B/Aurora C and 100-fold more selective for Aurora A than 55 other kinases. Phase 2. | 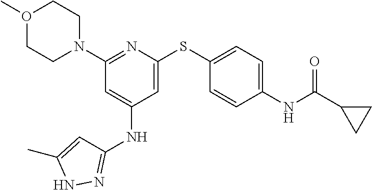 |
| Y-27632 2HCl | Autophagy, ROCK | Y-27632 2HCl is a selective ROCK1 (p160ROCK) inhibitor with Ki of 140 nM, exhibits >200-fold selectivity over other kinases, including PKC, cAMP-dependent protein kinase, MLCK and PAK. | 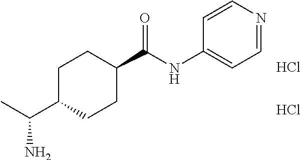 [SMILES: C[C@@H](N)[C@H]1CC[C@H](C(=O)Nc2ccncc2)CC1 ; Cl ; Cl] |
| Entinostat (MS-275) | HDAC | Entinostat (MS-275) strongly inhibits HDAC1 and HDAC3 with IC50 of 0.51 μM and 1.7 μM, compared with HDACs 4, 6, 8, and 10. Phase 1/2. | 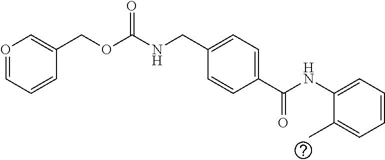 |
| Obatoclax Mesylate (GX15-070) | Bcl-2, Autophagy | Obatoclax (GX15-070) is an antagonist of Bcl-2 with Ki of 0.22 μM, can assist in overcoming MCL-1 mediated resistance to apoptosis. Phase 1/2. | 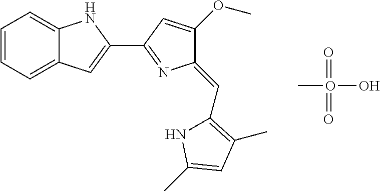 |
| Nutlin-3 | E3 Ligase, Mdm2 | Nutlin-3 is a potent and selective Mdm2 (RING finger-dependent ubiquitin protein ligase for itself and p53) antagonist with IC50 of 90 nM; stabilizes p73 in p53- deficient cells. | 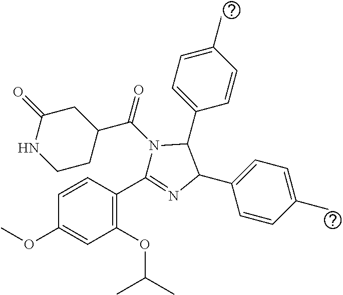 |
| Belinostat (PXD101) | HDAC | Belinostat (PXD101) is a novel HDAC inhibitor with IC50 of 27 nM, with activity demonstrated in cisplatin-resistant tumors. Phase 1/2. | 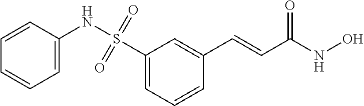 [SMILES: O=C(/C=C/c1cccc(S(=O)(=O)Nc2ccccc2)c1)NO] |
| PCI-24781 (Abexinostat) | HDAC | PCI-24781 is a novel pan-HDAC inhibitor mostly targeting HDAC1 with Ki of 7 nM, modest potent to HDACs 2, 3, 6, and 10 and greater than 40-fold selectivity against HDAC8. Phase 1/2. | 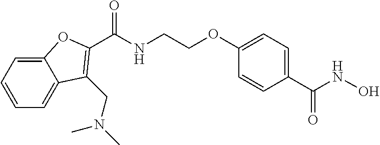 [SMILES: CN(C)Cc1c(C(=O)NCCOc2ccc(C(=O)NO)cc2)oc2ccccc12] |
| LAQ824 (Dacinostat) | HDAC | LAQ824 (Dacinostat) is a novel HDAC inhibitor with IC50 of 32 nM and is known to activate the p21 promoter. | 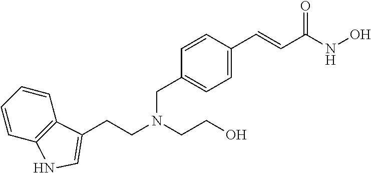 [SMILES: O=C(/C=C/c1ccc(CN(CCO)CCc2c[nH]c3ccccc23)cc1)NO] |
| Quisinostat (JNJ- 26481585) | HDAC | JNJ-26481585 is a novel second- generation HDAC inhibitor with highest potency for HDAC1 with IC50 of 0.11 nM, modest potent to HDACs 2, 4, 10, and 11; greater than 30-fold selectivity against HDACs 3, 5, 8, and 9 and lowest potency to HDACs 6 and 7. Phase 2. | 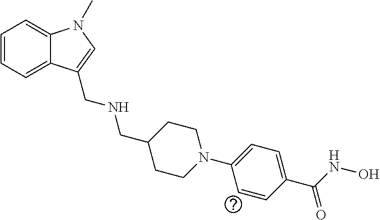 [SMILES: Cn1cc(CNCC2CCN(c3ccc(C(=O)NO)cc3)CC2)c2ccccc21] |
| MLN8054 | Aurora Kinase | MLN8054 is a potent and selective inhibitor of Aurora A with IC50 of 4 nM. It is more than 40-fold selective for Aurora A than Aurora B. Phase 1. | 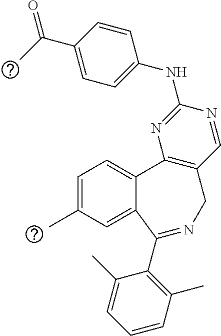 [SMILES: CC(=O)c1ccc(Nc2ncc3c(n2)-c2ccc(C)cc2C(c2c(C)cccc2C)=NC3)cc1] |
| ZM 447439 | Aurora Kinase | ZM 447439 is a selective and ATP- competitive inhibitor for Aurora A and Aurora B with IC50 of 110 nM and 130 nM, respectively. It is more than 8-fold selective for Aurora A/B than MEK1, Src, Lck and has little effect against CDK1/2/4, Plk1, Chk1, etc. | 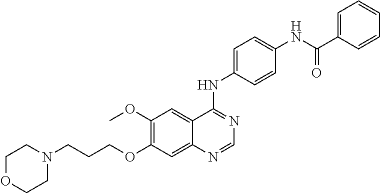 [SMILES: COc1cc2c(Nc3ccc(NC(=O)c4ccccc4)cc3)ncnc2cc1OCCCN1CCOCC1] |
| LY294002 | Autophagy, PI3K | LY294002 is the first synthetic molecule known to inhibit PI3Kα/δ/β with IC50 of 0.5 μM/0.57 μM/0.97 μM, respectively; more stable in solution than Wortmannin, and also blocks autophagosome formation. | 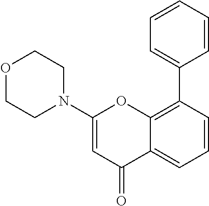 [SMILES: O=c1cc(N2CCOCC2)oc2c(-c3ccccc3)cccc12] |
| Danusertib (PHA- 739358) | c- RET, FG FR, Bcr- Abl, Aurora Kinase | Danusertib (PHA-739358) is an Aurora kinase inhibitor for Aurora A/B/C with IC50 of 13 nM/79 nM/61 nM, modestly potent to Abl, TrkA, c-RET and FGFR1, and less potent to Lck, VEGFR2/3, c-Kit, CDK2, etc. Phase 2. | 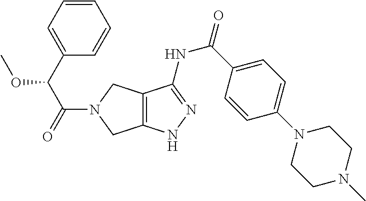 |
| Everolimus (RAD001) | mTOR | Everolimus (RAD001) is an mTOR inhibitor of FKBP12 with IC50 of 1.6-2.4 nM. | 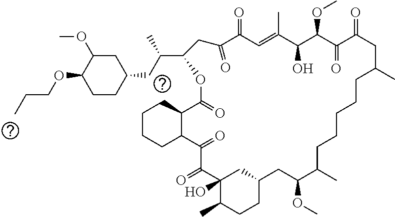 |
| Mocetinostat (MGCD0103) | HDAC | Mocetinostat (MGCD0103) is a potent HDAC inhibitor with most potency for HDAC1 with IC50 of 0.15 μM, 2- to 10- fold selectivity against HDAC2, 3, and 11, and no activity to HDAC4, 5, 6, 7, and 8. Phase 1/2. | 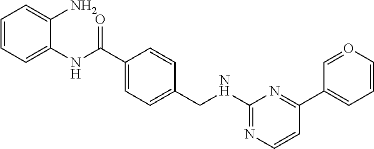 |
| SRT1720 | Sirtuin | SRT1720 is a selective SIRT1 activator with EC50 of 0.16 μM, but is >230-fold less potent for SIRT2 and SIRT3. | 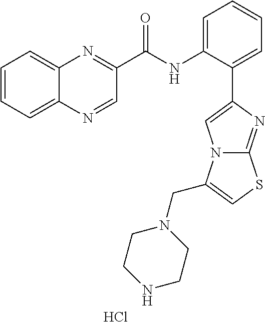 [SMILES: O=C(Nc1ccccc1-c1cn2c(CN3CCNCC3)csc2n1)c1cnc2ccccc2n1 ; Cl] |
| YM155 (Sepantronium Bromide) | Survivin | YM155 is a potent survivin suppressant by inhibiting Survivin promoter activity with IC50 of 0.54 nM; does not significantly inhibit SV40 promoter activity, but is observed to slightly inhibit the interaction of Survivin with XIAP. Phase 1/2. | 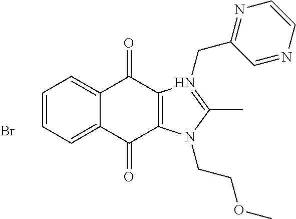 |
| Alisertib (MLN8237) | Aurora Kinase | Alisertib (MLN8237) is a selective Aurora A inhibitor with IC50 of 1.2 nM. It has >200- fold higher selectivity for Aurora A than Aurora B. Phase 3. | 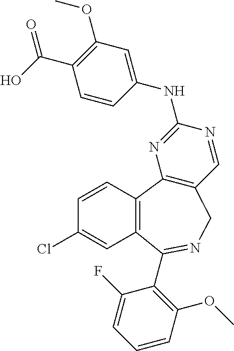 [SMILES: COc1cc(Nc2ncc3c(n2)-c2ccc(Cl)cc2C(c2c(F)cccc2OC)=NC3)ccc1C(=O)O] |
| AT9283 | JAK, Aurora Kinase, Bcr-Abl | AT9283 is a potent JAK2/3 inhibitor with IC50 of 1.2 nM/1.1 nM; also potent to Aurora A/B, Abl(T315I). Phase 1/2. | 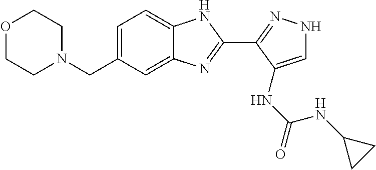 [SMILES: O=C(Nc1c[nH]nc1-c1nc2cc(CN3CCOCC3)ccc2[nH]1)NC1CC1] |
| Barasertib (AZD1152- HQPA) | Aurora Kinase | AZD1152-HQPA (Barasertib) is a highly selective Aurora B inhibitor with IC50 of 0.37 nM, ~100 fold more selective for Aurora B over Aurora A. | 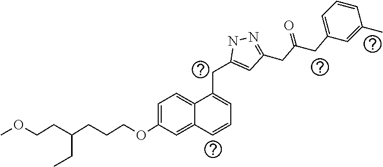 |
| Paclitaxel | Microtubule Associated, Autophagy | Paclitaxel is a microtubule polymer stabilizer with IC50 of 0.1 pM in human endothelial cells. | 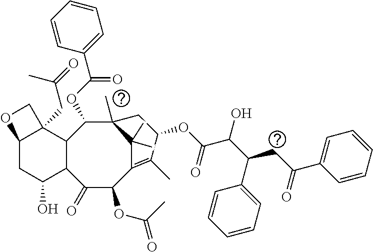 [SMILES: CC(=O)C[C@@]12CO[C@@H]1C[C@@H](O)C1C(=O)[C@H](OC(C)=O)C3=C(C)[C@@H](OC(=O)C(O)[C@@H](CC(=O)c4ccccc4)c4ccccc4)C[C@@](C)([C@@H](OC(=O)c4ccccc4)C12)C3(C)C] |
| SNS-314 Mesylate | Aurora Kinase | SNS-314 Mesylate is a potent and selective inhibitor of Aurora A, Aurora B and Aurora C with IC50 of 9 nM, 31 nM, and 3 nM, respectively. It is less potent to Trk A/B, Flt4, Fms, Axl, c-Raf and DDR2. Phase 1. | 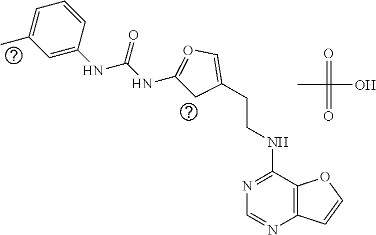 |
| CEP-18770 (Delanzomib) | Proteasome | CEP-18770 is an orally active inhibitor of the chymotrypsin-like activity of proteasome with IC50 of 3.8 nM, with only marginal inhibition of the tryptic and peptidylglutamyl activities of the proteosome. Phase 1/2. | 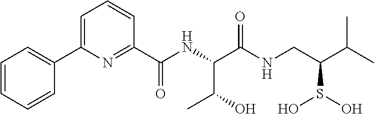 |
| Valproic acid sodium salt (Sodium valproate) | Autophaqy, HDAC, GABA Receptor | Valproic acid sodium salt (Sodium valproate) is a HDAC inhibitor with IC50 of 0.4 mM and also inhibits GABA- transaminase or succinic semialdehyde dehydrogenase. | 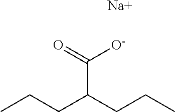 |
| CYC116 | Aurora Kinase, VEGFR | CYC116 is a potent inhibitor of Aurora A/B with Ki of 8.0 nM/9.2 nM, is less potent to VEGFR2 (Ki of 44 nM), with 50-fold greater potency than CDKs, not active against PKA, Akt/PKB, PKC, no effect on GSK- 3α/β, CK2, Plk1 and SAPK2A. Phase 1. | 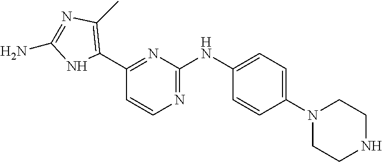 [SMILES: Cc1nc(N)[nH]c1-c1ccnc(Nc2ccc(N3CCNCC3)cc2)n1] |
| JNJ- | p53, E3 | JNJ-26854165 acts as a HDM2 ubiquitin | |
| 26854165 | Ligase | ligase antagonist and also induces early | |
| (Serdemetan) | | apoptosis in p53 wild-type cells, inhibits | |
| | | cellular proliferation followed by delayed | |
| | | apoptosis in the absence of functional p53. | |
| | | Phase 1. | |
| ENMD-2076 | Aurora Kinase, FLT3, VRGFR | ENMD-2076 has selective activity against Aurora A and Flt3 with IC50 of 14 nM and 1.86 nM, 25-fold selective for Aurora A than over Aurora B and less potent to VEGFR2/KDR and VEGFR3, FGFR1 and FGFR2 and PDGFRα Phase 2. | 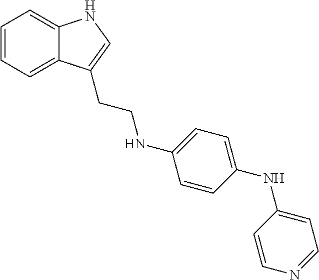 [SMILES: c1ccc2c(CCNc3ccc(Nc4ccncc4)cc3)c[nH]c2c1] |
| Thalidomide | TNF- alpha, E3 Ligase | Thalidomide was introduced as a sedative drug, immunomodulatory agent and also is investigated for treating symptoms of many cancers. Thalidomide inhibits an E3 ubiquitin ligase, which is a CRBN-DDB1- Cul4A complex. Phase 3. | 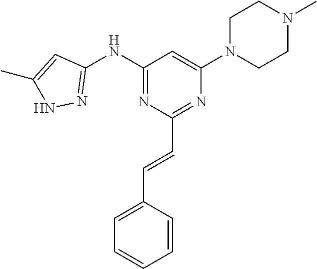 |
| CUDC-101 | HDAC, HER2, EGFR | CUDC-101 is a potent multi-targeted inhibitor against HDAC, EGFR and HER2 with IC50 of 4.4 nM, 2.4 nM, and 15.7 nM, and inhibits class I/II HDACs, but not class III, Sir-type HDACs. Phase 1. | 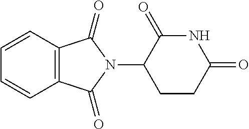 [SMILES: O=C1CCC(N2C(=O)c3ccccc3C2=O)C(=O)N1] |
| Doxorubicin (Adriamycin) | Autophagy, Topoisomerase | Doxorubicin (Adriamycin) is an antibiotic agent that inhibits DNA topoisomerase II and induces DNA damage and apoptosis. | 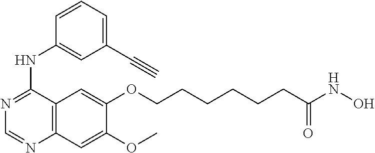 [SMILES: C#Cc1cccc(Nc2ncnc3cc(OC)c(OCCCCCCC(=O)NO)cc23)c1] |
| PFI-1 (PF- 6405761) | Epigenetic Reader Domain | PFI-1 is a selective BET (bromodomain- containing protein) inhibitor for BRD4 with IC50 of 0.22 μM. | 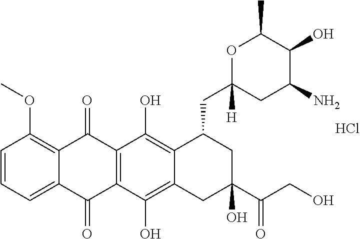 [SMILES: COc1cccc2c1C(=O)c1c(O)c3c(c(O)c1C2=O)C[C@@](O)(C(=O)CO)C[C@H]3C[C@H]1C[C@H](N)[C@H](O)[C@H](C)O1 ; Cl] |
| KU- 0063794 | mTOR | KU-0063794 is a potent and highly specific dual-mTOR inhibitor of mTORC1 and mTORC2 with IC50 of ~10 nM; no effect on PI3Ks. | 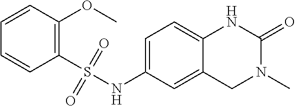 [SMILES: COc1ccccc1S(=O)(=O)Nc1ccc2c(c1)CN(C)C(=O)N2] |
| 2- Methoxy- estradiol (2-MeOE2) | HIF | 2-Methoxyestradiol depolymerizes microtubules and blocks HIF-1α nuclear accumulation and HIF-transcriptional activity. Phase 2. | 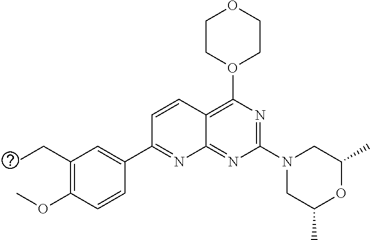 |
| Temozolomide | Autophagy | Temozolomide is a DNA damage inducer. | 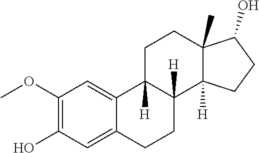 |
| Vincristine | Microtubule Associated, Autophagy | Vincristine is an inhibitor of polymerization of microtubules by binding to tubulin with IC50 of 32 μM. | 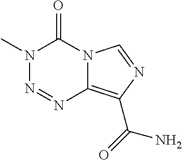 |
| JNJ- 7706621 | CDK, Aurora Kinase | JNJ-7706621 is pan-CDK inhibitor with the highest potency on CDK1/2 with IC50 of 9 nM/4 nM and showing >6-fold selectivity for CDK1/2 than CDK3/4/6. It also potently inhibits Aurora A/B and has no activity on Plk1 and Wee1. | 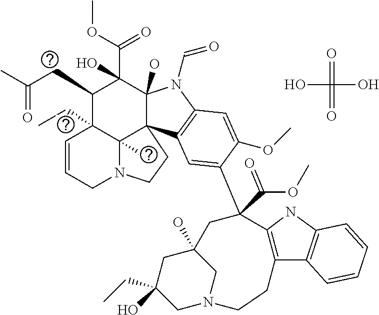 |
| WYE-354 | mTOR | WYE-354 is a potent, specific and ATP- competitive inhibitor of mTOR with IC50 of 5 nM, blocks mTORC1/P-S6K(T389) and mTORC2/P-AKT(S473) not P-AKT(1308), selective for mTOR than PI3Kα (>100-fold) and PI3Kγ (>500-fold). | 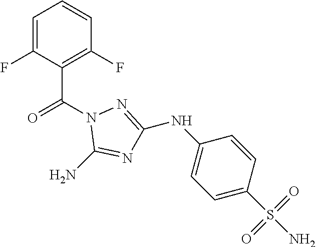 [SMILES: Nc1nc(Nc2ccc(S(N)(=O)=O)cc2)nn1C(=O)c1c(F)cccc1F] |
| Cilnidipine | Calcium Channel | Cilnidipine is a calcium channel blocker. | 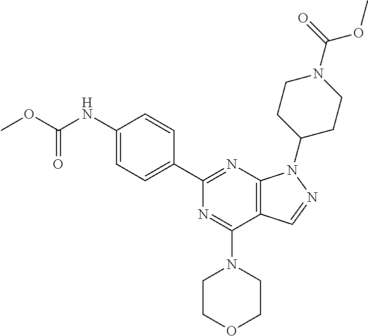 |
| Dexametha- sone (DHAP) | Autophagy, IL Receptor | Dexamethasone is an anti-inflammatory and immunosuppressant. Glucocorticoidan. | 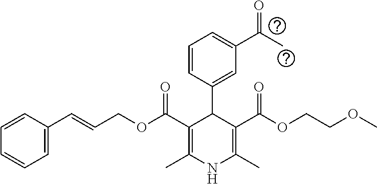 [SMILES: COCCOC(=O)C1=C(C)NC(C)=C(C(=O)OC/C=C/c2ccccc2)C1c1cccc(C(C)=O)c1] |
| Nafamostat Mesylate | Proteasome | Nafamostat Mesylate is an anticoagulant. Phase 4. | 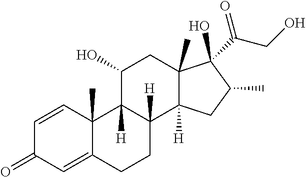 |
| Omeprazole | Autophagy, Proton Pump | Omeprazole(Prilosec) is a proton pump inhibitor used in the treatment of dyspepsia. | 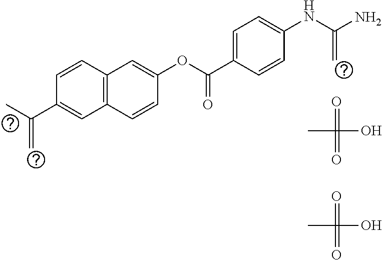 |
| Resveratrol | Sirtuin, Autophagy | Resveratrol is a phytoalexin produced naturally by several plants with anti-cancer, anti-inflammatory, blood-sugar-lowering and other beneficial cardiovascular effects. | 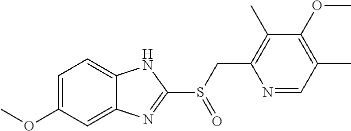 |
| Droxinostat | HDAC | Droxinostat (CMH, 5809354) is a selective inhibitor of HDAC, mostly for HDACs 6 and 8 with IC50 of 2.47 μM and 1.46 μM, greater than 8-fold selective against HDAC3 and no inhibition to HDAC1, 2, 4, 5, 7, 9, and 10. | 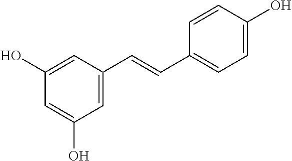 [SMILES: Oc1ccc(/C=C/c2cc(O)cc(O)c2)cc1] |
| Ranolazine 2HCl | Calcium Channel | Ranolazine 2HCl, is an antianginal medication. | 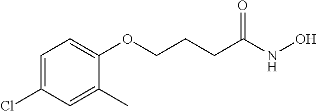 [SMILES: Cc1cc(Cl)ccc1OCCCC(=O)NO] |
| Aurora A Inhibitor I | Aurora Kinase | Aurora A Inhibitor I is a novel, potent, and selective inhibitor of Aurora A with IC50 of 3.4 nM. It is 1000-fold more selective for Aurora A than Aurora B. | 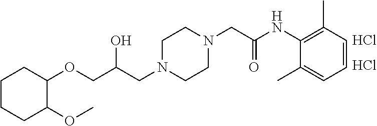 [SMILES: COC1CCCCC1OCC(O)CN1CCN(CC(=O)Nc2c(C)cccc2C)CC1 ; Cl ; Cl] |
| PHA- 680632 | Aurora Kinase | PHA-680632 is potent inhibitor of Aurora A, Aurora B and Aurora C with IC50 of 27 nM, 135 nM and 120 nM, respectively. It has 10- to 200-fold higher IC50 for FGFR1, FLT3, LCK, PLK1, STLK2, and VEGFR2/3. | 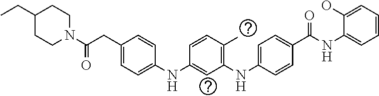 [SMILES: CCC1CCN(C(=O)Cc2ccc(Nc3ccc(C)c(Nc4ccc(C(=O)Nc5ccccc5O)cc4)c3)cc2)CC1] |
| MC1568 | HDAC | MC1568 is a selective HDAC inhibitor for maize HD1-A with IC50 of 100 nM. It is 34- fold more selective for HD1-A than HD1-B. | 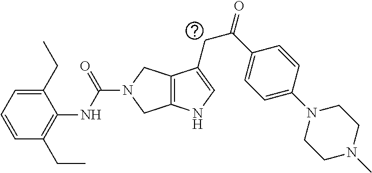 [SMILES: CCc1cccc(CC)c1NC(=O)N1Cc2[nH]cc(CC(=O)c3ccc(N4CCN(C)CC4)cc3)c2C1] |
| Pracinostat (SB939) | HDAC | SB939 is a potent pan-HDAC inhibitor with IC50 of 40-140 nM with exception for HDAC6. It has no activity against the class III isoenzyme SIRT I. Phase 2. | 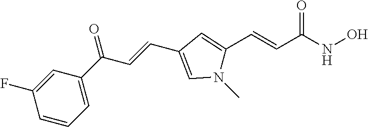 [SMILES: Cn1cc(/C=C/C(=O)c2cccc(F)c2)cc1/C=C/C(=O)NO] |
| CCT129202 | Aurora Kinase | CCT129202 is an ATP-competitive pan- Aurora inhibitor for Aurora A, Aurora B and Aurora C with IC50 of 0.042 μM, 0.198 μM and 0.227 μM, respectively. It is less potent to FGFR3, GSK3β, PDGFRβ, etc. | 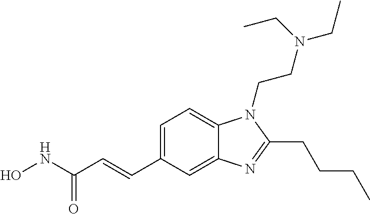 [SMILES: CCCCc1nc2cc(/C=C/C(=O)NO)ccc2n1CCN(CC)CC] |
| SAR245409 (XL765) | PI3K, mTOR | SAR245409 (XL765) is a dual inhibitor of mTOR/PI3K, mostly for p110γ with IC50 of 9 nM; also inhibits DNA-PK and mTOR. Phase 1/2. | 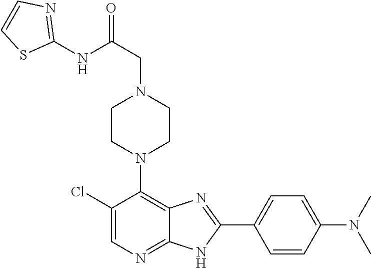 [SMILES: CN(C)c1ccc(-c2nc3c(N4CCN(CC(=O)Nc5nccs5)CC4)c(Cl)cnc3[nH]2)cc1] |
| Hesperadin | Aurora Kinase | Hesperadin potently inhibits Aurora B with IC50 of 250 nM. It markedly reduces the activity of AMPK, Lck, MKK1, MAPKAP-K1, CHK1 and PHK while it does not inhibit MKK1 activity in vivo. | 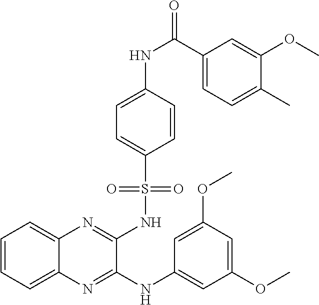 [SMILES: COc1cc(Nc2nc3ccccc3nc2NS(=O)(=O)c2ccc(NC(=O)c3ccc(C)c(OC)c3)cc2)cc(OC)c1] |
| EX 527 (Selisistat) | Sirtuin | EX 527 is a potent and selective SIRT1 inhibitor with IC50 of 38 nM, exhibits >200- fold selectivity against SIRT2 and SIRT3. | 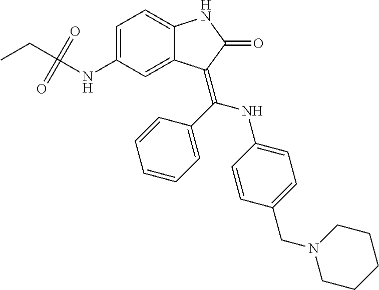 |
| AZD8055 | mTOR | AZD8055 is a novel ATP-competitive mTOR inhibitor with IC50 of 0.8 nM with excellent selectivity (~1,000-fold) against PI3K isoforms and ATM/DNA-PK. Phase 1. | 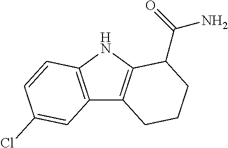 [SMILES: NC(=O)C1CCCc2c1[nH]c1ccc(Cl)cc21] |
| Fasudil (HA-1077) HCl | ROCK, Autophagy | Fasudil (HA-1077) is a potent inhibitor of ROCK-II, PKA, PKG, PKC, and MLCK with Ki of 0.33 μM, 1.6 μM, 1.6 μM, 3.3 μM and 36 μM, respectively. | 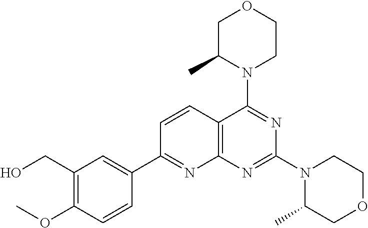 [SMILES: COc1ccc(-c2ccc3c(N4CCOC[C@@H]4C)nc(N4CCOC[C@@H]4C)nc3n2)cc1CO] |
| Isradipine | Calcium Channel | Isradipine(Dynacirc) is a calcium channel blocker with an IC50 of 34 ± 8 μM. | 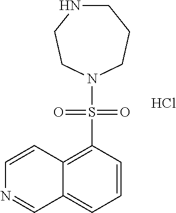 [SMILES: O=S(=O)(c1cccc2cnccc12)N1CCCNCC1 ; Cl] |
| Carbamaze- pine | Autophagy, Sodium Channel | Carbamazepine (Carbatrol) is a sodium channel blocker with IC50 of 131 μM in rat brain synaptosomes. | 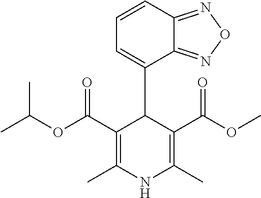 [SMILES: COC(=O)C1=C(C)NC(C)=C(C(=O)OC(C)C)C1c1cccc2nonc12] |
| Divalproex Sodium | Autophagy | Divalproex sodium consists of a compound of sodium valproate and valproic acid in a 1:1 molar relationship in an enteric coated form. | 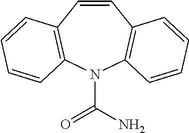 [SMILES: NC(=O)N1c2ccccc2C=Cc2ccccc21] |
| Gemcitabine | Autophagy DNA/RNA Synthesis | Gemcitabine(Gemzar) belongs to the group of medicines called antimetabolites. Phase 3. | 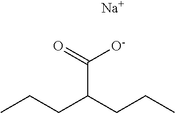 |
| | | | 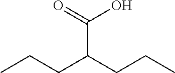 |
| Nimodipine | Calcium Channel, Autophagy | Nimodipine(Nimotop) is a dihydropyridine derivative and an analogue of the calcium Autophagy channel blocker nifedipine, with antihypertensive activity. Nimodipine decreases intracellular free Ca2+, Beclin-1 and autophagy. | 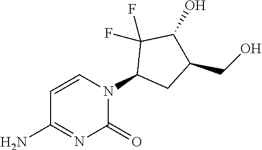 [SMILES: Nc1ccn([C@@H]2C[C@H](CO)[C@@H](O)C2(F)F)c(=O)n1] |
| Azithromycin | Autophagy | Azithromycin is an antibiotic for inhibition of parasite growth with IC50 of 8.4 μM. | 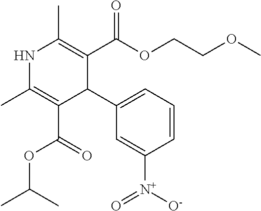 |
| Felodipine | Calcium Channel | Felodipine is a selective L-type Ca2+ channel blocker with IC50 of 0.15 nM. | 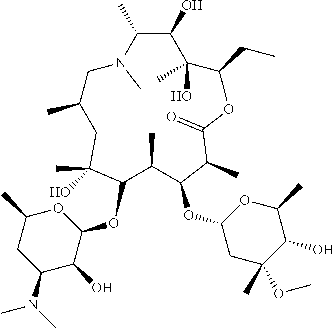 |
| Amlodipine | Calcium Channel | Amlodipine(Norvasc) is a long-acting calcium channel blocker with an IC50 of 1.9 nM. | 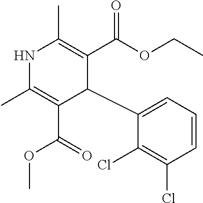 [SMILES: CCOC(=O)C1=C(C)NC(C)=C(C(=O)OC)C1c1cccc(Cl)c1Cl] |
| Tamoxifen Citrate | Estrogen/ progestogen Receptor, Autophagy | Tamoxifen Citrate is an antagonist of the estrogen receptor by competitive inhibition of estrogen binding. | 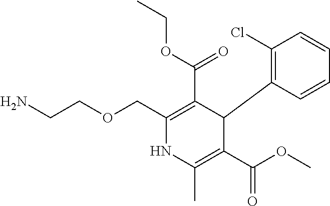 [SMILES: CCOC(=O)C1=C(COCCN)NC(C)=C(C(=O)OC)C1c1ccccc1Cl] |
| Amiodarone HCl | Autophagy, Potasium Channel | Amiodarone HCl is an antiarrhythmic drug for inhibition of ATP-sensitive potassium channel with IC50 of 19.1 μM. | 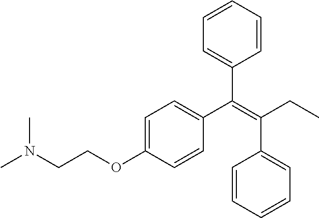 [SMILES: CC/C(=C(\c1ccccc1)c1ccc(OCCN(C)C)cc1)c1ccccc1] |
| | | | 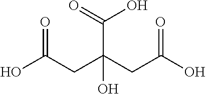 [SMILES: O=C(O)CC(O)(CC(=O)O)C(=O)O] |
| Lacidipine | Calcium Channel | Lacidipine (Lacipil, Motens) is a L-type calcium channel blocker. | 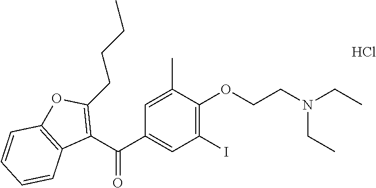 [SMILES: CCCCc1oc2ccccc2c1C(=O)c1cc(C)c(OCCN(CC)CC)c(I)c1 ; Cl] |
| PCI-34051 | HDAC | PCI-34051 is a potent and specific HDAC8 inhibitor with IC50 of 10 nM. It has greater than 200-fold selectivity over HDAC1 and 6, more than 1000-fold selectivity over HDAC2, 3, and 10. | 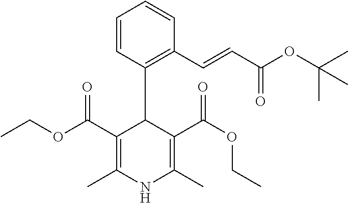 [SMILES: CCOC(=O)C1=C(C)NC(C)=C(C(=O)OCC)C1c1ccccc1/C=C/C(=O)OC(C)(C)C] |
| Flunarizine 2HCl | Calcium Channel | Flunarizine dihydrochloride is a dihydrochloride salt form which is a calcium channel blocker with a Ki of 68 nM. | 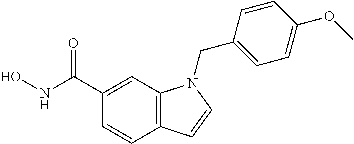 [SMILES: COc1ccc(Cn2ccc3ccc(C(=O)NO)cc32)cc1] |
| Clevidipine Butyrate | Calcium Channel | Cleviprex (Clevidipine) is a dihydropyridine calcium channel blocker use as agent for the reduction of blood pressure. | 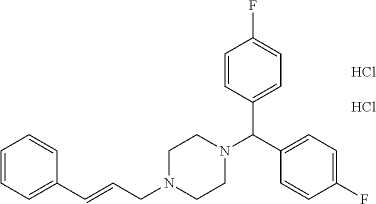 [SMILES: Fc1ccc(C(c2ccc(F)cc2)N2CCN(C/C=C/c3ccccc3)CC2)cc1 ; Cl ; Cl] |
| Gabexate Mesylate | Proteasome | Gabexate is a serine protease inhibitor with IC50 of 0.19 μM which is used therapeutically in the treatment of pancreatitis and disseminated intravascular coagulation. | 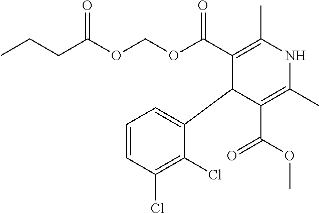 [SMILES: CCCC(=O)OCOC(=O)C1=C(C)NC(C)=C(C(=O)OC)C1c1cccc(Cl)c1Cl] |
| KW-2449 | Aurora Kinase, Bcr- Abl, FLT3 | KW-2449 is a multiple-targeted inhibitor, mostly for Flt3 with IC50 of 6.6 nM, modestly potent to FGFR1, Bcr-Abl and Aurora A; little effect on PDGFIRβ, IGF-1R, EGFR. Phase 1. | 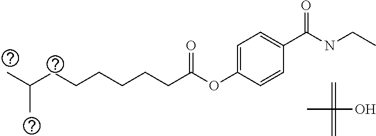 |
| Givinostat (ITF2357) | HDAC | Givinostat (ITF2357) is a potent HDAC inhibitor for HDAC2, HDAC1B and HDAC1A with IC50 of 10 nM, 7.5 nM and 16 nM. Phase 1/2. | 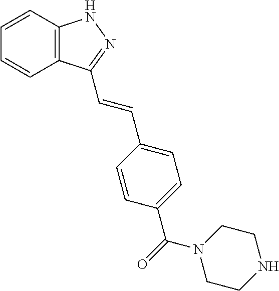 [SMILES: O=C(c1ccc(/C=C/c2n[nH]c3ccccc23)cc1)N1CCNCC1] |
| MLN2238 | Proteasome | MLN2238 inhibits the chymotrypsin-like proteolytic (β5) site of the 20S proteasome with IC50 and Ki of 3.4 nM and 0.93 nM, respectively, also inhibits the caspase-like (β1) and trypsin-like (β2) proteolytic sites, with IC50 of 31 and 3500 nM. | 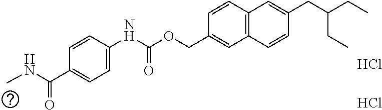 [SMILES: CCC(CC)Cc1ccc2cc(COC(=O)Nc3ccc(C(=O)NC)cc3)ccc2c1 ; Cl ; Cl] |
| MLN9708 | Proteasome | MLN9708 immediately hydrolyzed to MLN2238, the biologically active form, on exposure to aqueous solutions or plasma. MLN2238 inhibits the chymotrypsin-like proteolytic (β5) site of the 20S proteasome with IC50/Ki of 3.4 nM/0.93 nM, less potent to β1 and little activity to βB2. Phase 3. | 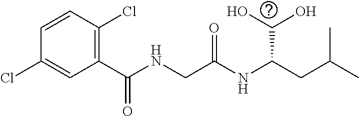 [SMILES: CC(C)C[C@H](NC(=O)CNC(=O)c1cc(Cl)ccc1Cl)C(O)O] |
| SGI-1776 free base | Pim | SGI-1776 is a novel ATP competitive inhibitor of Pim1 with IC50 of 7 nM, 50- and 10-fold selective versus Pim2 and Pim3, also potent to Flt3 and haspin. Phase 1. | 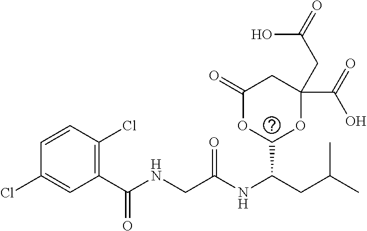 [SMILES: CC(C)C[C@H](NC(=O)CNC(=O)c1cc(Cl)ccc1Cl)C1OC(=O)CC(CC(=O)O)(C(=O)O)O1] |
| PP242 | mTOR, Autophagy | PP242 is a selective mTOR inhibitor with IC50 of 8 nM; targets both mTOR complexes with >10- and 100-fold selectivity for mTOR than PI3Kδ or PI3Kα/β/γ, respectively. | 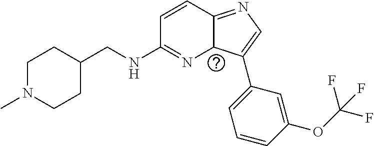 [SMILES: CN1CCC(CNC2=NC3C(c4cccc(OC(F)(F)F)c4)=CN=C3C=C2)CC1] |
| TAME | E3 Ligase, APC | Tosyl-L-Arginine Methyl Ester (TAME) is an APC inhibitor. | 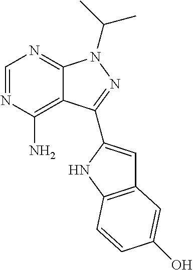 [SMILES: CC(C)n1nc(-c2cc3cc(O)ccc3[nH]2)c2c(N)ncnc21] |
| Degrasyn (WP1130) | DUB, Bcr-Abl | WP1130 (Degrasyn) is a selective deubiquitinase (DUB: USP5, UCH-L1, USP9x, USP14, and UCH37) inhibitor and also suppresses Bcr/Abl, also a JAK2 transducer (without affecting 20S proteasome) and activator of transcription (STAT). | 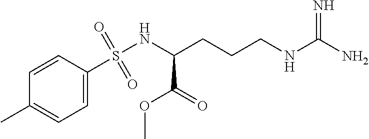 [SMILES: COC(=O)[C@H](CCCNC(=N)N)NS(=O)(=O)c1ccc(C)cc1] |
| AR-42 | HDAC | AR-42 is an HDAC inhibitor with IC50 30 nM. | 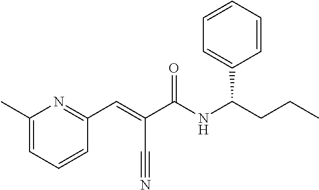 [SMILES: CCC[C@H](NC(=O)/C(C#N)=C/c1cccc(C)n1)c1ccccc1] |
| (-)- Parthenolide | E3 Ligase | (-)-Parthenolide is a sesquiterpene lactone which occurs naturally in the plant feverfew(Tanacetum parthenium) and also promotes the ubiquitination of MDM2 and activates p53 cellular functions. | 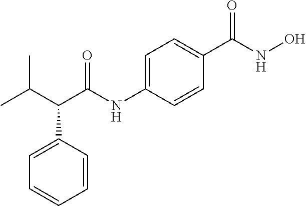 [SMILES: CC(C)[C@H](C(=O)Nc1ccc(C(=O)NO)cc1)c1ccccc1] |
| Tetrandrine | Calcium Channel | Tetrandrine, a bis-benzylisoquinoline alkaloid derived from Stephania tetrandra, is a calcium channel blocker. | 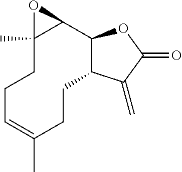 [SMILES: C=C1C(=O)O[C@H]2[C@H]1CC/C(C)=C\CC[C@@]1(C)O[C@@H]21] |
| Chrysophanic Acid | mTOR, EGFR | Chrysophanic acid (Chrysophanol), a natural anthraquinone isolated from Dianella longifolia, is a EGFR/mTOR pathway inhibitor. | 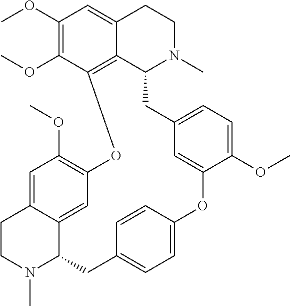 |
| Rotundine | Dopamine Receptor | Rotundine (L-tetrahydropalmatine, L-THP) is a selective dopamine D1 receptor antagonist with IC50 of 166 nM. | 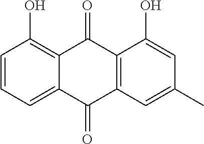 [SMILES: Cc1cc(O)c2c(c1)C(=O)c1cccc(O)c1C2=O] |
| Forskolin | cAMP | Forskolin is a ubiquitous activator of eukaryotic adenylyl cyclase (AC), commonly used to raise levels of cAMP in the study and research of cell physiology. | 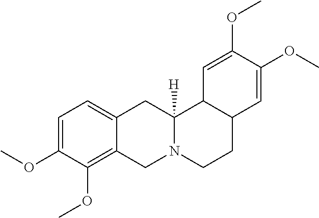 |
| Bupivacaine HCl | cAMP | Bupivacaine hydrochloride(Marcain) is a more potent cAMP production inhibitor with an IC50 of 2.3 μM | 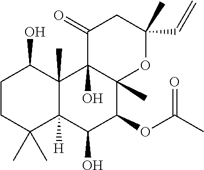 |
| Clonidine HCl | Adrenergic Receptor, Autophagy | Clonidine hydrochloride(Catapres) is a direct-acting α2 adrenergic agonist with an ED50 of 0.02 ± 0.01 mg/kg. | 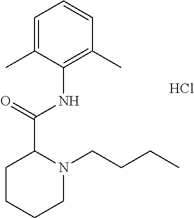 [SMILES: CCCCN1CCCCC1C(=O)Nc1c(C)cccc1C ; Cl] |
| Loperamide HCl | Opioid Receptor, Autophagy | Loperamide HCl is an opioid-receptor agonist with an ED50 of 0.15 mg/kg. | 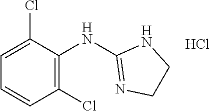 [SMILES: Clc1cccc(Cl)c1NC1=NCCN1 ; Cl] |
| Manidipine | Calcium Channel | Manidipine (Manyper) is a lipophilic, third- generation, highly vasoselective dihydropyridine calcium antagonist with an IC50 of 2.6 nM. | 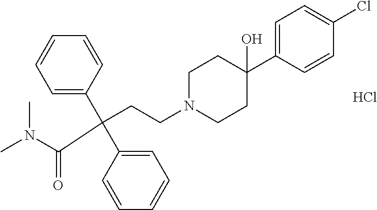 [SMILES: CN(C)C(=O)C(CCN1CCC(O)(c2ccc(Cl)cc2)CC1)(c1ccccc1)c1ccccc1 ; Cl] |
| Manidipine 2HCl | Calcium Channel | Manidipine 2HCl is a HCl salt form of Manidipine, which is a calcium channel blocker with IC50 of 2.6 nM, used clinically as an antihypertensive. Phase 4. | 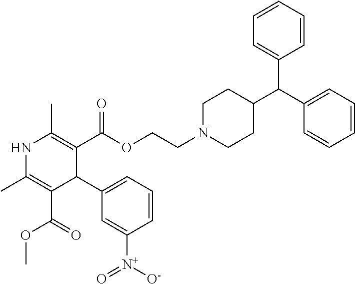 [SMILES: COC(=O)C1=C(C)NC(C)=C(C(=O)OCCN2CCC(C(c3ccccc3)c3ccccc3)CC2)C1c1cccc([N+](=O)[O-])c1] |
| Nitrendipine | Calcium Channel, Autophagy | Nitrendipine is a dihydropyridine calcium channel blocker with an IC50 of 95 nM. | 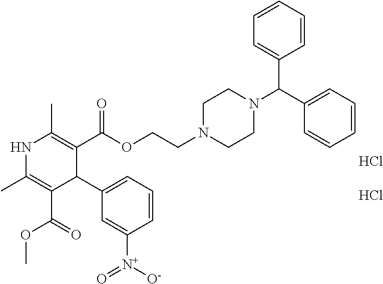 [SMILES: COC(=O)C1=C(C)NC(C)=C(C(=O)OCCN2CCN(C(c3ccccc3)c3ccccc3)CC2)C1c1cccc([N+](=O)[O-])c1 ; Cl ; Cl] |
| Tetracaine HCl | Calcium Channel | Tetracaine hydrochloride (Pontocaine) is a hydrochloride salt form of tetracaine which is a potent local anaesthetic and a channel function allosteric inhibitor. | 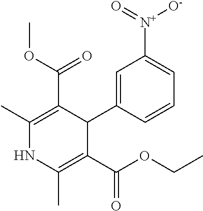 [SMILES: CCOC(=O)C1=C(C)NC(C)=C(C(=O)OC)C1c1cccc([N+](=O)[O-])c1] |
| MG-132 | Proteasome | MG-132 is an inhibitor of proteasome with IC50 of 100 nM, and also inhibits calpain with IC50 of 1.2 μM. | 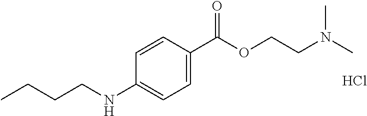 [SMILES: CCCCNc1ccc(C(=O)OCCN(C)C)cc1 ; Cl] |
| PP121 | DNA- PK, PDGFR, mTOR | PP-121 is a multi-targeted inhibitor of PDGFR, Hck, mTOR, VEGFR2, Src and Abl with IC50 of 2 nM, 8 nM, 10 nM, 12 nM, 14 nM and 18 nM, also inhibits DNA-PK with IC50 of 60 nM. | 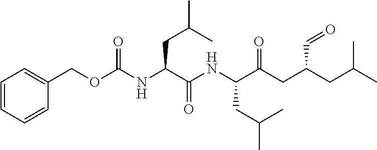 [SMILES: CC(C)C[C@@H](C=O)CC(=O)[C@H](CC(C)C)NC(=O)[C@H](CC(C)C)NC(=O)OCc1ccccc1] |
| OSI-027 | mTOR | OSI-027 is a selective and potent dual inhibitor of mTORC1 and mTORC2 with IC50 of 22 nM and 65 nM, and more than 100-fold selectivity observed for mTOR than PI3Kα, PI3Kβ, PI3Kγ or DNA-PK. | 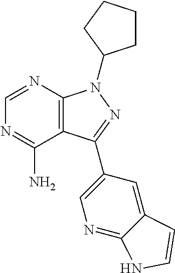 [SMILES: Nc1ncnc2c1c(-c1cnc3[nH]ccc3c1)nn2C1CCCC1] |
| Tubastatin A HCl | HDAC | Tubastatin A is a potent and selective HDAC6 inhibitor with IC50 of 15 nM. It is selective (1000-fold more) against all other isozymes except HDAC8 (57-fold more). | 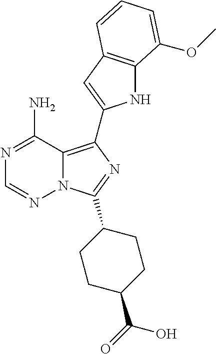 [SMILES: COc1cccc2cc(-c3nc([C@H]4CC[C@H](C(=O)O)CC4)n4ncnc(N)c34)[nH]c12] |
| PF- 05212384 (PKI-587) | mTOR, PI3K | PKI-587 is a highly potent dual inhibitor of PI3Kα, PI3Kγ and mTOR with IC50 of 0.4 nM, 5.4 nM and 1.6 nM, respectively. Phase 2. | 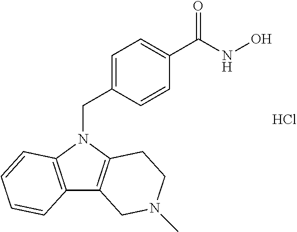 [SMILES: CN1CCc2c(c3ccccc3n2Cc2ccc(C(=O)NO)cc2)C1 ; Cl] |
| GSK2126458 (GSK458) | PI3K, mTOR | GSK2126458 is a highly selective and potent inhibitor of p110α/β/δ/γ, mTORC1/2 with Ki of 0.019 nM/0.13 nM/0.024 nM/0.06 nM and 0.18 nM/0.3 nM, respectively. Phase 1. | 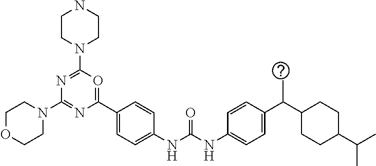 |
| WYE- 125132 (WYE-132) | mTOR | WYE-125132 is a highly potent, ATP- competitive mTOR inhibitor with IC50 of 0.19 nM; highly selective for mTOR versus PI3Ks or PI3K-related kinases hSMG1 and ATR. | 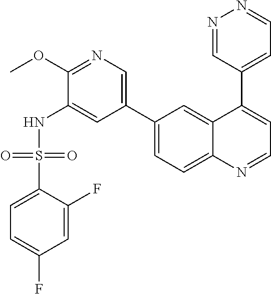 [SMILES: COc1ncc(-c2ccc3nccc(-c4ccnnc4)c3c2)cc1NS(=O)(=O)c1ccc(F)cc1F] |
| Geldanamycin | HSP (e.g. HSP90), Autophagy | Geldanamycin is a natural existing HSP90 inhibitor with Kd of 1.2 μM, specifically disrupts glucocorticoid receptor (GR)/HSP association. | 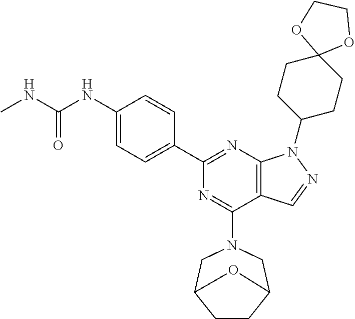 [SMILES: CNC(=O)Nc1ccc(-c2nc(N3CC4CCC(C3)O4)c3cnn(C4CCC5(CC4)OCCO5)c3n2)cc1] |
| TAK-901 | Aurora Kinase | TAK-901 is a novel inhibitor of Aurora A/B with IC50 of 21 nM/15 nM. It is not a potent inhibitor of cellular JAK2, c-Src or Abl. Phase 1. | 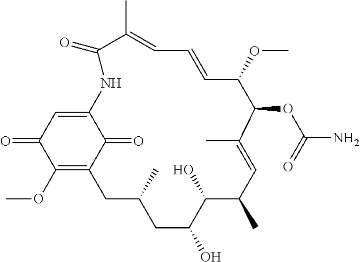 [SMILES: COC1=C2C[C@H](C)C[C@@H](O)[C@H](O)[C@H](C)/C=C(\C)[C@H](OC(N)=O)[C@@H](OC)/C=C/C=C(\C)C(=O)NC(=CC1=O)C2=O] |
| AMG-900 | Aurora Kinase | AMG 900 is a potent and highly selective pan-Aurora kinases inhibitor for Aurora A/B/C with IC50 of 5 nM/4 nM /1 nM. It is >10-fold selective for Aurora kinases than p38α, Tyk2, JNK2, Met and Tie2. Phase 1. | 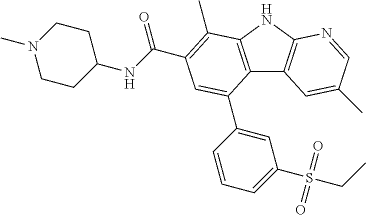 [SMILES: CCS(=O)(=O)c1cccc(-c2cc(C(=O)NC3CCN(C)CC3)c(C)c3[nH]c4ncc(C)cc4c23)c1] |
| Nilvadipine | Calcium Channel | Nilvadipine is a potent calcium channel blocker with an IC50 of 0.03 nM. | 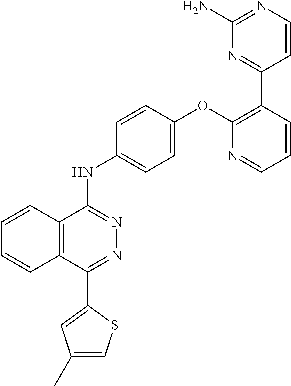 [SMILES: Cc1csc(-c2nnc(Nc3ccc(Oc4ncccc4-c4ccnc(N)n4)cc3)c3ccccc23)c1] |
| GSK1070916 | Aurora Kinase | GSK1070916 is a reversible and ATP- competitive inhibitor of Aurora B/C with IC50 of 3.5 nM/6.5 nM. It displays >100-fold selectivity against the closely related Aurora A-TPX2 complex. Phase 1. | 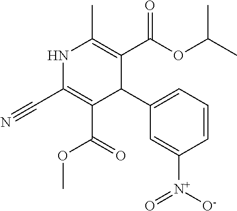 [SMILES: COC(=O)C1=C(C#N)NC(C)=C(C(=O)OC(C)C)C1c1cccc([N+](=O)[O-])c1] |
| PF- 04691502 | Akt, mTOR, PI3K | PF-04691502 is an ATP-competitive PI3K(α/β/δ/γ)/mTOR dual inhibitor with Ki of 1.8 nM/2.1 nM/1.6 nM/1.9 nM and 16 nM, little activity against either Vps34, AKT, PDK1, p70S6K, MEK, ERK, p38, or JNK. Phase 2. | 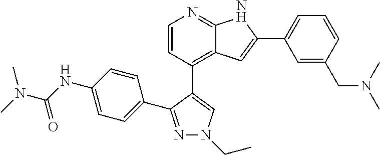 |
| CCT137690 | Aurora Kinase | CCT137690 is a highly selective inhibitor of Aurora A, Aurora B and Aurora C with IC50 of 15 nM, 25 nM and 19 nM. It has little effect on hERG ion-channel. | 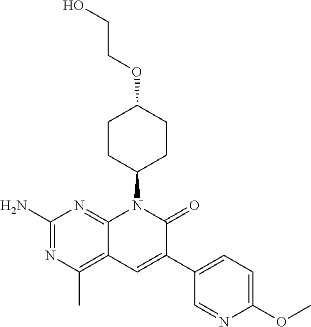 [SMILES: COc1ccc(-c2cc3c(C)nc(N)nc3n([C@H]3CC[C@H](OCCO)CC3)c2=O)cn1] |
| BGT226 (NVP- BGT226) | PI3K, mTOR | NVP-BGT226 is a novel class I PI3K/mTOR inhibitor for PI3Kα/β/γ with IC50 of 4 nM/63 nM/38 nM. Phase 1/2. | 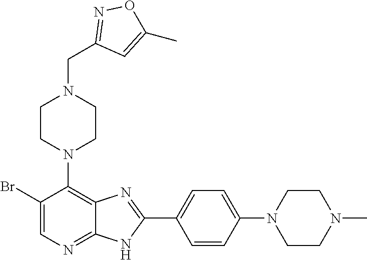 [SMILES: Cc1cc(CN2CCN(c3c(Br)cnc4[nH]c(-c5ccc(N6CCN(C)CC6)cc5)nc34)CC2)no1] |
| Wortmannin | Autophagy, ATM/ ATR, PI3K | Wortmannin is a PI3K with IC50 of 3 nM, first described PI3K inhibitors, but little selectivity within the PI3K family. Also blocks autophagosome formation and potently inhibits DNA-PK/ATM with IC50 of 16 nM and 150 nM. Phase 4. | 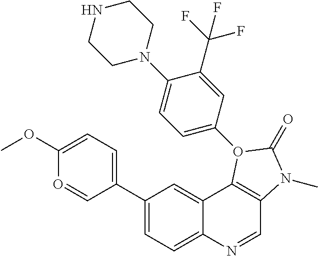 |
| | | | 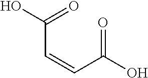 |
| CUDC-907 | PI3K, HDAC | CUDC-907 is a dual PI3K and HDAC inhibitor for PI3Kα and HDAC1/2/3/10 with IC50 of 19 nM and 1.7 nM/5 nM/1.8 nM/2.8 nM, respectively. Phase 1. | 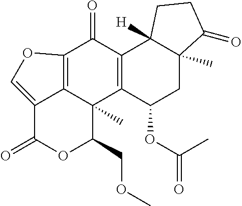 [SMILES: COC[C@H]1OC(=O)c2coc3c2[C@@]1(C)C1=C(C3=O)[C@@H]2CCC(=O)[C@@]2(C)C[C@@H]1OC(C)=O] |
| 3- Methyladenine | Autophagy, PI3K | 3-Methyladenine is a selective PI3K inhibitor for Vps34 and PI3Kγ with IC50 of 25 μM and 60 μM; blocks class I PI3K consistently, whereas suppression of class III PI3K is transient, and also blocks autophagosome formation. | 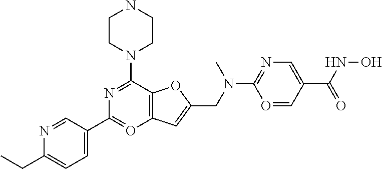 |
| MK-5108 (VX-689) | Aurora Kinase | MK-5108 (VX-689) is a highly selective Aurora A inhibitor with IC50 of 0.064 nM and is 220- and 190-fold more selective for Aurora A than Aurora B/C, while it inhibits TrkA with less than 100-fold selectivity. Phase 1. | 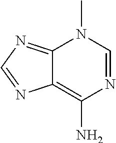 |
| Nocodazole | Microtubule Associated, Autophagy | Nocodazole is a rapidly-reversible inhibitor of microtubule polymerization, also inhibits Abl, Abl(E255K) and Abl(T315I) with IC50 of 0.21 μM, 0.53 μM and 0.64 μM, respectively. | 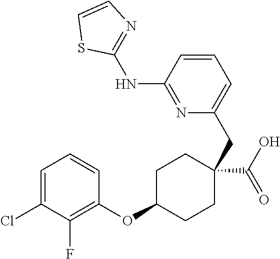 [SMILES: O=C(O)[C@]1(Cc2cccc(Nc3nccs3)n2)CC[C@@H](Oc2cccc(Cl)c2F)CC1] |
| M344 | HDAC | M344 is a potent HDAC inhibitor with IC50 of 100 nM and able to induce cell differentiation. | 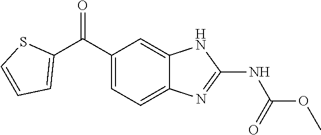 [SMILES: COC(=O)Nc1nc2ccc(C(=O)c3cccs3)cc2[nH]1] |
| RITA (NSC 652287) | E3 Ligase, p53 | RITA (NSC 652287) induces both DNA- protein and DNA-DNA cross-links with no detectable DNA single-strand breaks, and also inhibits MDM2-p53 interaction by targeting p53. | 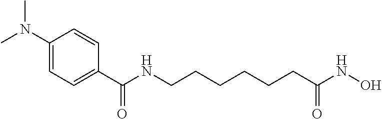 [SMILES: CN(C)c1ccc(C(=O)NCCCCCCC(=O)NO)cc1] |
| Sirtinol | Sirtuin | Sirtinol is a specific SIRT1 and SIRT2 inhibitor with IC50 of 131 μM and 38 μM, respectively. | 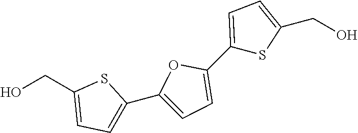 [SMILES: OCc1ccc(-c2ccc(-c3ccc(CO)s3)o2)s1] |
| Torin 2 | ATM/ ATR, mTOR | Torin 2 is a potent and selective mTOR inhibitor with IC50 of 0.25 nM; 800-fold greater selectivity for mTOR than PI3K and improved pharmacokinetic properties. Inhibition of ATM/ATR/DNA-PK with EC50 of 28 nM/35 nM/118 nM, respectively. | 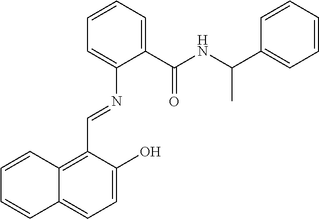 [SMILES: CC(NC(=O)c1ccccc1/N=C/c1c(O)ccc2ccccc12)c1ccccc1] |
| CI994 (Tacedinaline) | HDAC | CI-994 (Tacedinaline) is an anti-cancer drug which inhibits HDAC1 with IC50 of 0.57 μM and causes G1 cell cycle arrest. Phase 3. | 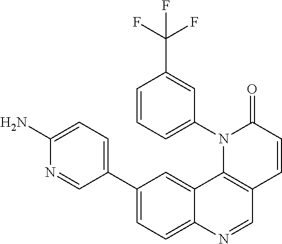 [SMILES: Nc1ccc(-c2ccc3ncc4ccc(=O)n(-c5cccc(C(F)(F)F)c5)c4c3c2)cn1] |
| Torin 1 | Autophagy, mTOR | Torin 1 is a potent inhibitor of mTORC1/2 with IC50 of 2 nM/10 nM; exhibits 1000-fold selectivity for mTOR than PI3K. | 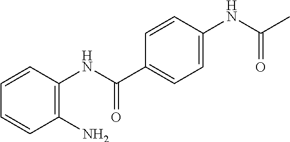 [SMILES: CC(=O)Nc1ccc(C(=O)Nc2ccccc2N)cc1] |
| Carfilzomib (PR-171) | Proteasome | Carfilzomib (PR-171) is an irreversible proteasome inhibitor with IC50 of <5 nM, displayed preferential in vitro inhibitory potency against the ChT-L activity in the β5 subunit, but little or no effect on the PGPH and T-L activities. | 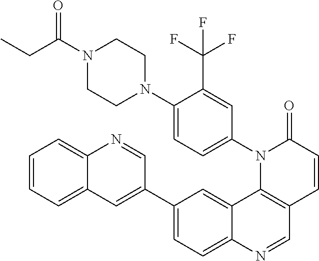 [SMILES: CCC(=O)N1CCN(c2ccc(-n3c(=O)ccc4cnc5ccc(-c6cnc7ccccc7c6)cc5c43)cc2C(F)(F)F)CC1] |
| BAY 11- 7082 | IκB/IKK, E2 conjugating | BAY 11-7082 is a NF-κB inhibitor, inhibits TNFα-induced IκBα phosphorylation with IC50 of 10 μM. | 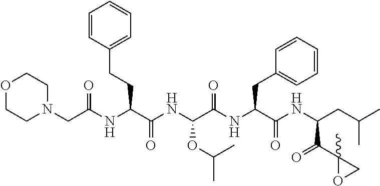 [SMILES: CC(C)C[C@H](NC(=O)[C@H](Cc1ccccc1)NC(=O)[C@@H](NC(=O)[C@H](CCc1ccccc1)NC(=O)CN1CCOCC1)OC(C)C)C(=O)C1(C)CO1] |
| IOX2 | HIF | IOX2 is a potent inhibitor of HIF-1α prolyl hydroxylase-2 (PHD2) with IC50 of 21 nM, >100-fold selectivity over JMJD2A, JMJD2C, JMJD2E, JMJD3, or the 2OG oxygenase FIH. | 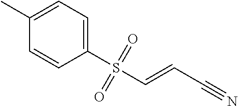 |
| Pifithrin-α (PFTα) | p53, Autophagy | Pifithrin-α is an inhibitor of p53, inhibiting p53-dependent transactivation of p53- responsive genes. | 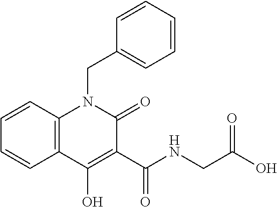 [SMILES: O=C(O)CNC(=O)c1c(O)c2ccccc2n(Cc2ccccc2)c1=O] |
| Aspirin | Proteasome | Aspirin is a salicylate drug, often used as an analgesic to relieve minor aches and pains, as an antipyretic to reduce fever, and as an anti-inflammatory medication. | 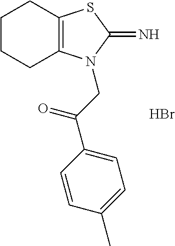 [SMILES: Cc1ccc(C(=O)Cn2c3c(sc2=N)CCCC3)cc1 ; Br] |
| Azelnidipine | Calcium Channel | Azelnidipine is a dihydropyridine calcium channel blocker. | 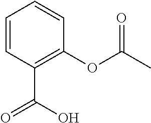 |
| Trifluoperazine 2HCl | Autophagy | Trifluoperazine is a dopamine D2 receptor inhibitor with IC50 of 1.1 nM. | 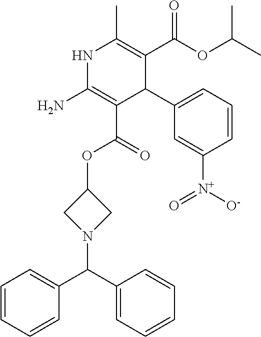 [SMILES: CC1=C(C(=O)OC(C)C)C(c2cccc([N+](=O)[O-])c2)C(C(=O)OC2CN(C(c3ccccc3)c3ccccc3)C2)=C(N)N1] |
| Sulfacetamide Sodium | Autophagy | Sulfacetamide Sodium is an anti-biotic. | 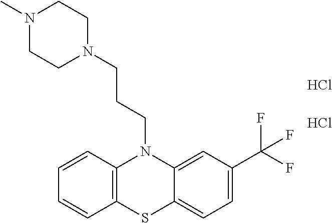 [SMILES: CN1CCN(CCCN2c3ccccc3Sc3ccc(C(F)(F)F)cc32)CC1 ; Cl ; Cl] |
| Sodium Phenyl- butyrate | HDAC | Sodium Phenylbutyrate is a transcriptional regulators that act by altering chromatin structure via the modulation of HDAC activity. | 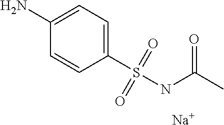 |
| Brefeldin A | ATPase, Autophagy | Brefeldin A is a lactone antibiotic and ATPase inhibitor for protein transport with IC50 of 0.2 μM in HCT 116 cells, induces cancer cell differentiation and apoptosis. | 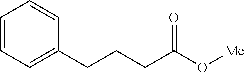 [SMILES: COC(=O)CCCc1ccccc1] |
| Oprozomib (ONX 0912) | Proteasome | Oprozomib (ONX 0912) is an orally bioavailable inhibitor for CT-L activity of 20S proteasome β5/LMP7 with IC50 of 36 nM/82 nM. | 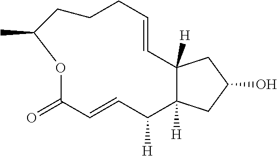 |
| PYR-41 | E1 Activating | PYR-41 is the first cell-permeable inhibitor of ubiquitin-activating enzyme E1, with no activity at E2. | 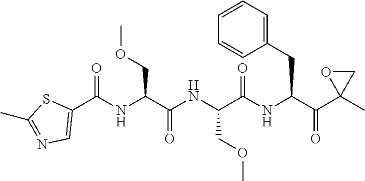 |
| PR-619 | DUB | PR-619 is a non-selective, reversible inhibitor of the deubiquitinylating enzymes (DUBs) with EC50 of 1-20 μM. | 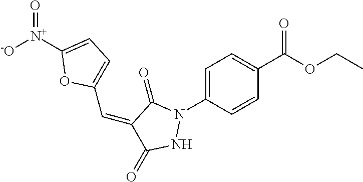 |
| P5091 (P005091) | DUB | P5091(P005091) is a selective and potent inhibitor of ubiquitin-specific protease 7 (USP7) with EC50 of 4.2 μM and the closely related USP47. | 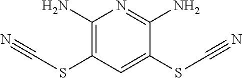 [SMILES: N#CSc1cc(SC#N)c(N)nc1N] |
| P22077 | DUB | P22077 is an inhibitor of ubiquitin-specific protease USP7 with EC50 of 8.6 μM, also inhibits the closely related USP47. | 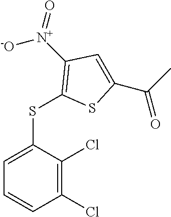 |
| IU1 | DUB | IU2 is a cell-permeable, reversible and selective proteasome inhibitor of human USP14 with IC50 of 4.7 μM, 25-fold selective to IsoT. | 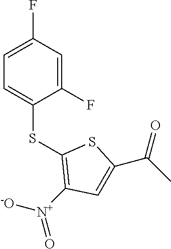 |
| LDN-57444 | DUB | LDN-57444 is a reversible, competitive proteasome inhibitor for Uch-L1 with IC50 of 0.88 μM, 28-fold selectivity over isoform Uch-L3. | 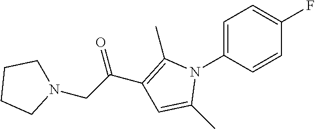 [SMILES: Cc1cc(C(=O)CN2CCCC2)c(C)n1-c1ccc(F)cc1] |
| TCID | DUB | TCID is a DUB inhibitor for ubiquitin C- terminal hydrolase L3 with IC50 of 0.6 μM, 125-fold selective to L1. | 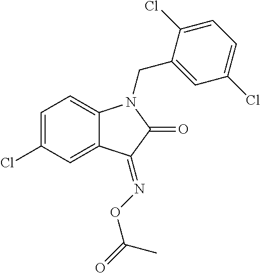 [SMILES: CC(=O)O/N=C1/C(=O)N(Cc2cc(Cl)ccc2Cl)c2ccc(Cl)cc21] |
| ONX-0914 (PR-957) | Proteasome | ONX-0914 (PR-957) is a potent and selective immunoproteasome inhibitor with minimal cross-reactivity for the constitutive proteasome. | 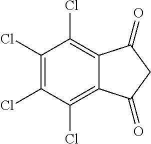 [SMILES: O=C1CC(=O)c2c(Cl)c(Cl)c(Cl)c(Cl)c21] |
| DBeQ | p97 | DBeQ is a selective, potent, reversible, and ATP-competitive p97 inhibitor with IC50 of 1.5 μM. | 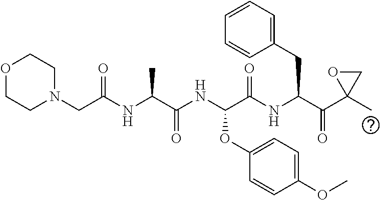 [SMILES: COc1ccc(O[C@H](NC(=O)[C@H](C)NC(=O)CN2CCOCC2)C(=O)N[C@@H](Cc2ccccc2)C(=O)C2(C)CO2)cc1] |
| NMS-873 | p97 | NMS-873is an allosteric and specific p97 inhibitor with IC50 of 30 nM. | 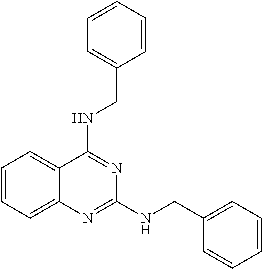 [SMILES: c1ccc(CNc2nc(NCc3ccccc3)c3ccccc3n2)cc1] |
| Tenovin-1 | p53, E3 Ligase | Tenovin-1 protects against MDM2-mediated p53 degradation, which involves ubiquitination, and acts through inhibition of protein-deacetylating activities of SirT1 and SirT2. | 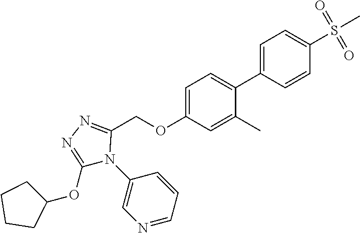 [SMILES: Cc1cc(OCc2nnc(OC3CCCC3)n2-c2cccnc2)ccc1-c1ccc(S(C)(=O)=O)cc1] |
| Rocilinostat (ACY-1215) | HDAC | Rocilinostat (ACY-1215) is a selective HDAC6 inhibitor with IC50 of 5 nM. It is >10-fold more selective for HDAC6 than HDAC1/2/3 (class I HDACs) with slight activity against HDAC8, minimal activity against HDAC4/5/7/9/11, Sirtuin1, and Sirtuin2. | 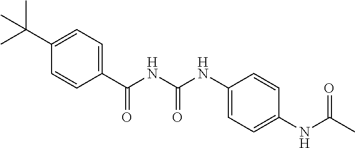 [SMILES: CC(=O)Nc1ccc(NC(=O)NC(=O)c2ccc(C(C)(C)C)cc2)cc1] |
| SMI-4a | Pim | SMI-4a is a potent inhibitor of Pim1 with IC50 of 17 nM, modest potent to Pim-2, does not significantly inhibit other serine/threonine- or tyrosine-kinases. | 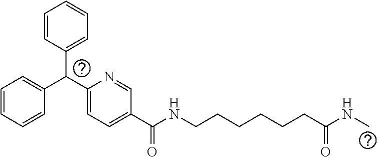 [SMILES: CNC(=O)CCCCCCNC(=O)c1ccc(C(c2ccccc2)c2ccccc2)nc1] |
| GDC-0349 | mTOR | GDC-0349 is a potent and selective ATP- competitive inhibitor of mTOR with Ki of 3.8 nM, 790-fold inhibitory effect against PI3Kα and other 266 kinases. Phase 1. | 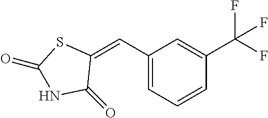 [SMILES: O=C1NC(=O)/C(=C\c2cccc(C(F)(F)F)c2)S1] |
| Scriptaid | HDAC | Scriptaid is an inhibitor of HDAC. It shows a greater effect on acetylated H4 than H3. | 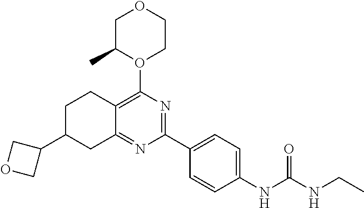 |

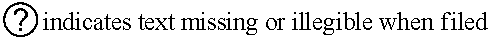

Other autophagy inhibiting agents can be selected from Pan-PI3K inhibitors, Vsp34 inhibitors, ULK inhibitors, or lysosome inhibitors selected from any one or more of the following:

| | Target of | |
|---|---|---|
| Inhibitor Name | Inhibitor | Structure |
| 3- methyladenine | Pan-PI3K | 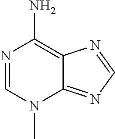 |
| Wortmannin | Pan-PI3K | 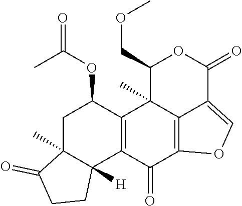 [SMILES: COC[C@H]1OC(=O)c2coc3c2[C@@]1(C)C1=C(C3=O)[C@@H]2CCC(=O)[C@@]2(C)C[C@H]1OC(C)=O] |
| LY294002 | Pan-PI3K | 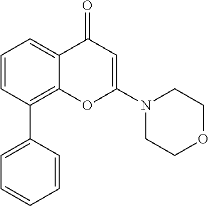 [SMILES: O=c1cc(N2CCOCC2)oc2c(-c3ccccc3)cccc12] |
| PT210* | Pan-PI3K | 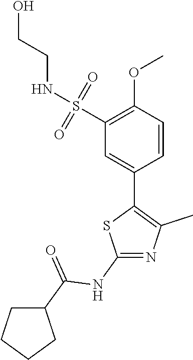 [SMILES: COc1ccc(-c2sc(NC(=O)C3CCCC3)nc2C)cc1S(=O)(=O)NCCO] |
| GSK- 2126458* | Pan-PI3K | 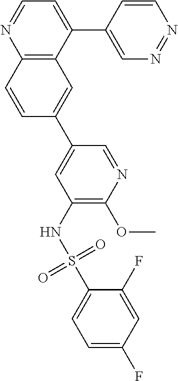 [SMILES: COc1ncc(-c2ccc3nccc(-c4ccnnc4)c3c2)cc1NS(=O)(=O)c1ccc(F)cc1F] |
| Spautin-1 | Vps34 | 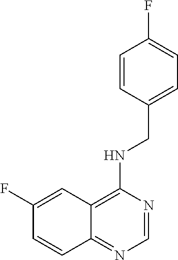 [SMILES: Fc1ccc(CNc2ncnc3ccc(F)cc23)cc1] |
| SAR405 | Vps34 | 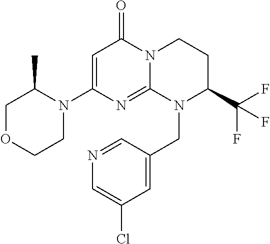 [SMILES: C[C@@H]1COCCN1c1cc(=O)n2c(n1)N(Cc1cncc(Cl)c1)[C@H](C(F)(F)F)CC2] |
| Compound 31 | Vps34 | 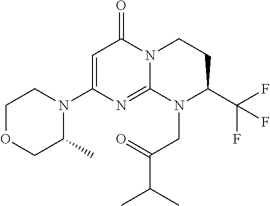 [SMILES: CC(C)C(=O)CN1c2nc(N3CCOC[C@H]3C)cc(=O)n2CC[C@H]1C(F)(F)F] |
| VPS34-IN1 | Vps34 | 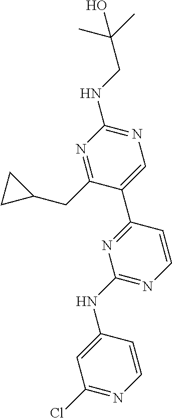 [SMILES: CC(C)(O)CNc1ncc(-c2ccnc(Nc3ccnc(Cl)c3)n2)c(CC2CC2)n1] |
| PIK-III | Vps34 | 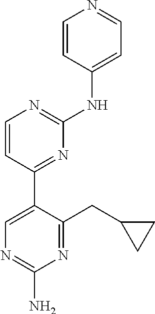 [SMILES: Nc1ncc(-c2ccnc(Nc3ccncc3)n2)c(CC2CC2)n1] |
| Compound 6 | ULK | 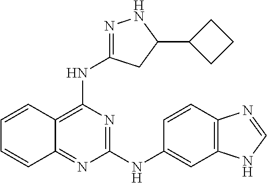 [SMILES: c1ccc2c(NC3=NNC(C4CCC4)C3)nc(Nc3ccc4nc[nH]c4c3)nc2c1] |
| MRT68921 | ULK | 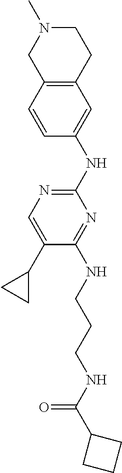 [SMILES: CN1CCc2cc(Nc3ncc(C4CC4)c(NCCCNC(=O)C4CCC4)n3)ccc2C1] |
| SBI-0206965 | ULK | 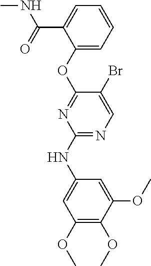 |
| Pepstatin A | Lysosome | 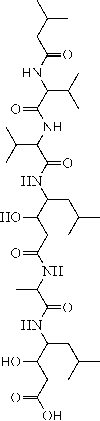 [SMILES: CC(C)CC(=O)NC(C(=O)NC(C(=O)NC(CC(C)C)C(O)CC(=O)NC(C)C(=O)NC(CC(C)C)C(O)CC(=O)O)C(C)C)C(C)C] |
| E64d | Lysosome | 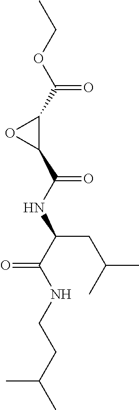 [SMILES: CCOC(=O)[C@H]1O[C@@H]1C(=O)N[C@@H](CC(C)C)C(=O)NCCC(C)C] |
| Bafilomycin A1 | Lysosome | 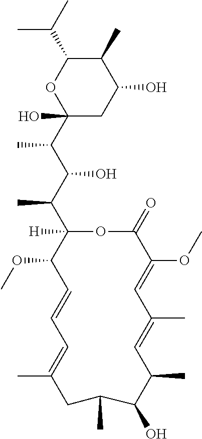 |
| Clomipramine | Lysosome | 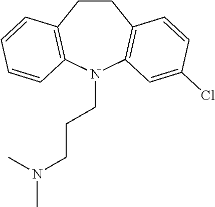 [SMILES: CN(C)CCCN1c2ccccc2CCc2ccc(Cl)cc21] |
| Lucanthone | Lysosome | 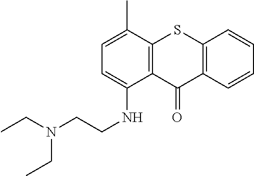 [SMILES: CCN(CC)CCNc1ccc(C)c2sc3ccccc3c(=O)c12] |
| Chloroquine | Lysosome | 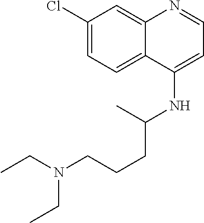 [SMILES: CCN(CC)CCCC(C)Nc1ccnc2cc(Cl)ccc12] |
| Hydroxy- chloro- quine | Lysosome | 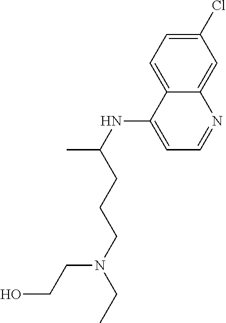 [SMILES: CCN(CCO)CCCC(C)Nc1ccnc2cc(Cl)ccc12] |
| Lys05 | Lysosome | 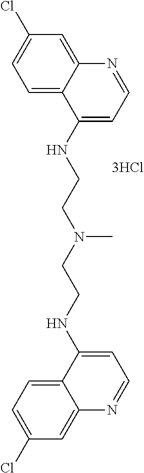 [SMILES: CN(CCNc1ccnc2cc(Cl)ccc12)CCNc1ccnc2cc(Cl)ccc12] |
| ARN5187 | Lysosome | 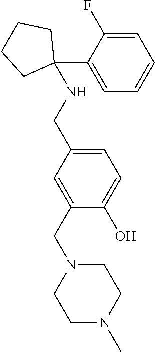 [SMILES: CN1CCN(Cc2cc(CNC3(c4ccccc4F)CCCC3)ccc2O)CC1] |
| Compound 30 | Lysosome | 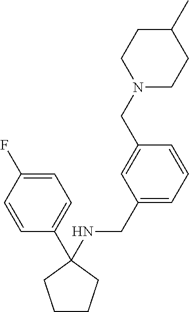 [SMILES: CC1CCN(Cc2cccc(CNC3(c4ccc(F)cc4)CCCC3)c2)CC1] |

Other inhibitors of autophagy that can be used can include known autophagy inhibitors, such as those listed below:

| Inhibitor | | |
|---|---|---|
| Name | Target | Structure |
| Concanamycin A | V-type (vacuolar) H+- ATPase inhibitor | 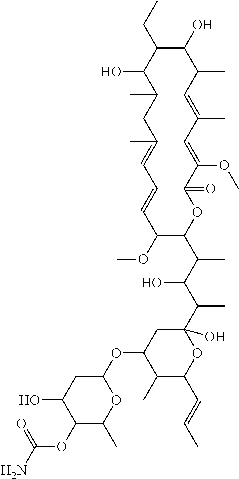 [SMILES: C/C=C/C1OC(O)(C(C)C(O)C(C)C2OC(=O)/C(OC)=C/C(C)=C/C(C)C(O)C(CC)C(O)C(C)C/C(C)=C/C=C/C2OC)CC(OC2CC(O)C(OC(N)=O)C(C)O2)C1C] |
| (±)-Bay K 8644 | L-type Ca2+- channel activator | 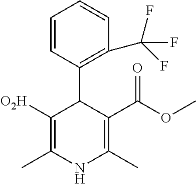 |
| FK 866 hydrochloride | NMPRTase inhibitor | 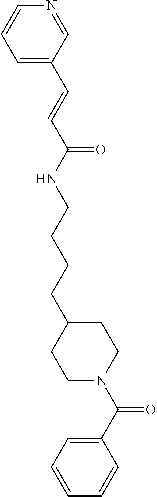 [SMILES: O=C(/C=C/c1cccnc1)NCCCCC1CCN(C(=O)c2ccccc2)CC1] |
| Xanthohumol | VCP inhibitor | 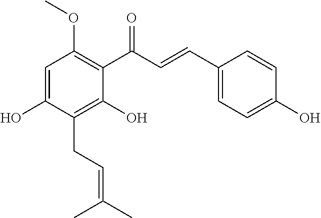 [SMILES: COc1cc(O)c(CC=C(C)C)c(O)c1C(=O)/C=C/c1ccc(O)cc1] |
| DBeQ | P97 ATPase inhibitor | 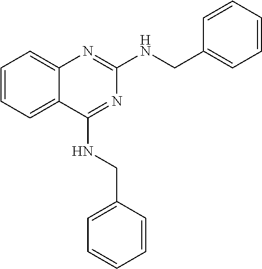 [SMILES: c1ccc(CNc2nc(NCc3ccccc3)c3ccccc3n2)cc1] |
| Omeprazole | H+, K+- ATPase inhibitor | 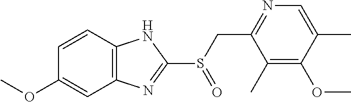 |
| Bafilomycin A1 | V-ATPase inhibitor, selective and reversible | 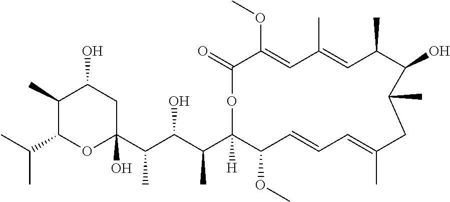 [SMILES: CO/C1=C\C(C)=C\[C@@H](C)[C@@H](O)[C@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O] |
| Chloroquine diphosphate | Antimalarial drug, TLR7 TLR9 inhibitor | 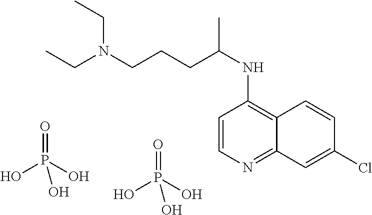 [SMILES: O=P(O)(O)O ; CCN(CC)CCCC(C)Nc1ccnc2cc(Cl)ccc12 ; O=P(O)(O)O] |
| KC01 | A selective inhibitor of ABHD16A | 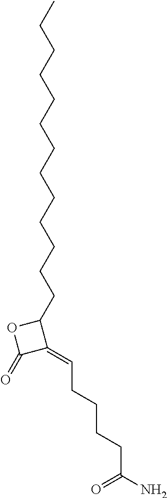 [SMILES: CCCCCCCCCCCCCC1OC(=O)/C1=C\CCCCC(N)=O] |
| SAR405 | Selective ATP- competitive inhibitor of Vps34 | 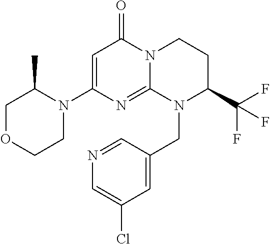 [SMILES: C[C@@H]1COCCN1c1cc(=O)n2c(n1)N(Cc1cncc(Cl)c1)[C@H](C(F)(F)F)CC2] |
| KC02 | Structural analog and inactive form of KC01 | 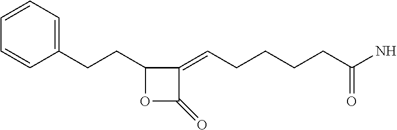 [SMILES: NC(=O)CCCC/C=C1\C(=O)OC1CCc1ccccc1] |
| LY3009120 | pan-RAF and RAF dimer inhibitor | 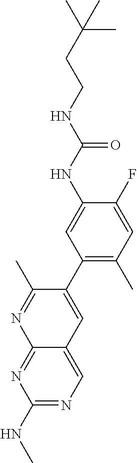 [SMILES: CNc1ncc2cc(-c3cc(NC(=O)NCCC(C)(C)C)c(F)cc3C)c(C)nc2n1] |
| SBI-0206965 | ULK1 inhibitor | 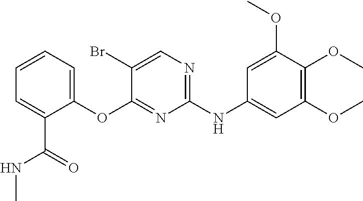 [SMILES: CNC(=O)c1ccccc1Oc1nc(Nc2cc(OC)c(OC)c(OC)c2)ncc1Br] |
| Spautin-1 | autophagy inhibitor | 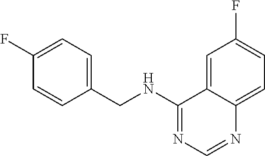 [SMILES: Fc1ccc(CNc2ncnc3ccc(F)cc23)cc1] |
| PIK-III | VPS34 inhibitor and inhibits autophagy | 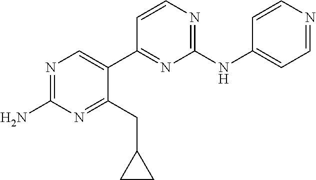 [SMILES: Nc1ncc(-c2ccnc(Nc3ccncc3)n2)c(CC2CC2)n1] |
| MRT68921 | dual autophagy kinase ULK1/2 inhibitor | 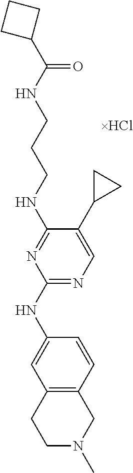 [SMILES: CN1CCc2cc(Nc3ncc(C4CC4)c(NCCCNC(=O)C4CCC4)n3)ccc2C1] |
| Pepstatin A | Aspartic proteinases inhibitor | 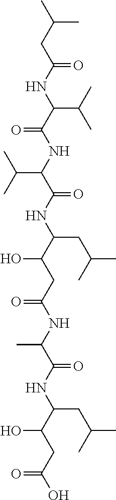 [SMILES: CC(C)CC(=O)NC(C(=O)NC(C(=O)NC(CC(C)C)C(O)CC(=O)NC(C)C(=O)NC(CC(C)C)C(O)CC(=O)O)C(C)C)C(C)C] |
| E 64d | Cysteine protease inhibitor | 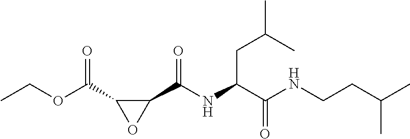 [SMILES: CCOC(=O)[C@H]1O[C@@H]1C(=O)N[C@@H](CC(C)C)C(=O)NCCC(C)C] |
| Paclitaxel (Taxol) | Antineoplastic agent | 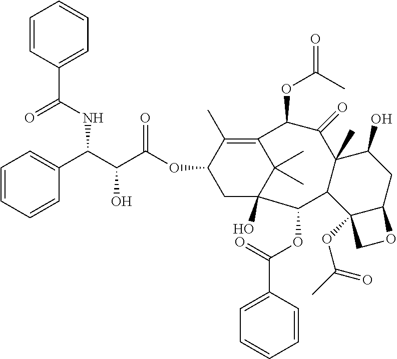 [SMILES: CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O] |
| Vinblastine sulfate | Anti-mitotic agent | 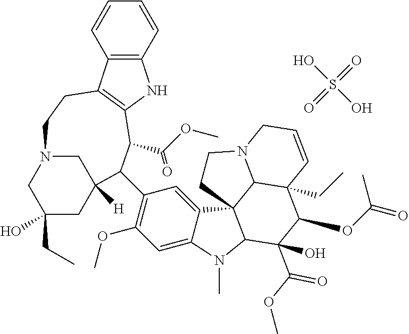 |
| Doxorubicin | Topo II inhibitor, immuno- suppresive antineoplastic antibiotic | 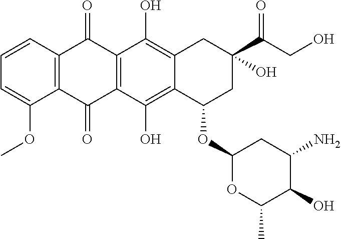 [SMILES: COc1cccc2c1C(=O)c1c(O)c3c(c(O)c1C2=O)C[C@@](O)(C(=O)CO)C[C@@H]3O[C@H]1C[C@H](N)[C@@H](O)[C@H](C)O1] |
| LY 294002 | Potent PI3K inhibitor | 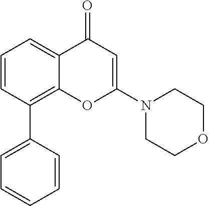 [SMILES: O=c1cc(N2CCOCC2)oc2c(-c3ccccc3)cccc12] |
| VPS34-IN1 | Vps34 inhibitor | 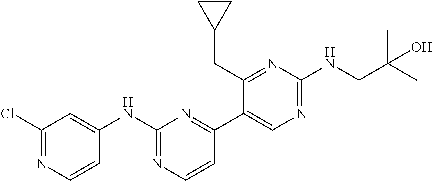 [SMILES: CC(C)(O)CNc1ncc(-c2ccnc(Nc3ccnc(Cl)c3)n2)c(CC2CC2)n1] |

An autophagy inhibiting agent, such as a ULK inhibitor can be used to inhibit autophagy, inhibit pregnancy, or provide contraception. For example, the present disclosure identified a ULK inhibitor, SBI-0206965. Other examples of ULK inhibitors can be: MRT68921 or LYN-1604.

As an example, the autophagy inhibiting agent or the ULK inhibiting agent can comprise

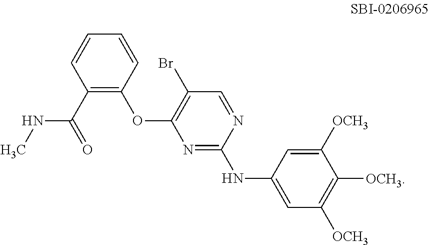 [SMILES: CNC(=O)c1ccccc1Oc1nc(Nc2cc(OC)c(OC)c(OC)c2)ncc1Br]

An autophagy inhibitor that can be used in the compositions and methods as described herein can be Zafirlukast. Zafirlukast is a leukotriene receptor antagonist. It blocks the receptor for leukotriene C4, D4, and E4. It can be used as an asthma medication. It increases migration of eosinophils and neutrophils, decreases adhesion of leukocytes, monocyte and neutrophil aggregation, and decreases airway edema, inflammation, capillary permeability and bronchoconstriction.

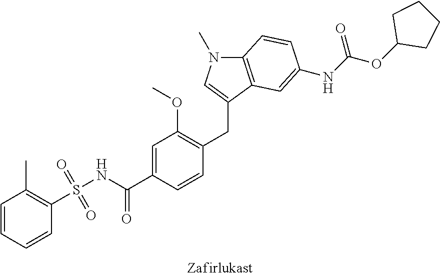 [SMILES: COc1cc(C(=O)NS(=O)(=O)c2ccccc2C)ccc1Cc1cn(C)c2ccc(NC(=O)OC3CCCC3)cc12]

Autophagy inhibiting agents can be used for treating a variety of diseases (see e.g., Rubinsztein et al. 2012 Nat Rev Drug Discov. 2012 September; 11(9): 709-730). For example, autophagy modulation can be a potential therapeutic for including metabolic conditions, neurodegenerative diseases, cancers, or infectious diseases.

Intrauterine System (IUS)

An intrauterine system (IUS), for example, an intrauterine device (IUD), can comprise compositions such as an autophagy inhibiting agent, as described herein.

Methods of making and compositions of IUSs are well known; see e.g. Dean et al., 2017, Intrauterine contraception: Devices, candidates, and selection, UpToDate; U.S. Pat. No. 7,252,839, incorporated herein by reference. Except as otherwise noted herein, therefore, the process of the present disclosure can be carried out in accordance with such processes.

The intrauterine device (IUD), also known as intrauterine contraceptive device (IUCD or ICD) or coil, can be a small, often T-shaped birth control device that is inserted into a woman's uterus to prevent pregnancy. An IUD, as described herein, can be one form of long-acting reversible birth control.

The intrauterine device comprising an autophagy inhibitor can be based on any of the below IUDs currently known in the art (e.g., by replacing the metal or the hormone with the autophagy inhibitor or a modified version thereof.

The types of intrauterine devices currently available, and the names they go by, differ by location. In the United States, there are currently only two types available: Non-hormonal copper IUD (ParaGard and others) and IUD with progestogen (Mirena and others). The WHO ATC labels both copper and hormonal devices as IUDs. In the United Kingdom, there are over 10 different types of copper IUDs available. In the UK, the term IUD refers only to these copper devices. Hormonal intrauterine contraception is considered to be a different type of birth control and is labeled with the term intrauterine system (IUS). In the US, five types of IUDs are available in the United States (US); one contains copper and four release levonorgestrel (LNg). Copper IUD containing 380 mm² copper, US Food and Drug Administration (FDA) approved for 10 years of use (abbreviated TCu380A, commercial name ParaGard). LNg-releasing IUD containing 52 mg LNg at initial placement and with an initial LNg release rate of 20 mcg/day, FDA approved for five years of use (abbreviated LNg52/5, commercial name Mirena). LNg-releasing IUD containing 52 mg LNg at initial placement and with an initial LNg release rate of 18.6 mcg/day, FDA approved for four years of use (abbreviated LNg52/4, commercial name Liletta). LNg-releasing IUD containing 19.5 mg LNg at initial placement with an initial LNg release rate of 17.5 mcg/day, FDA approved for up to 5 years of use (abbreviated LNg20/5, commercial name Kyleena). LNg-releasing IUD containing 13.5 mg LNg at initial placement and with an initial LNg release rate of 14 mcg/day, FDA approved for three years of use (abbreviated LNg14/3, commercial name Skyla).

In some embodiments, the autophagy inhibiting agent-releasing IUD can contain an autophagy inhibiting agent at initial placement of the IUD in an amount of about 0.01 mg; about 0.02 mg; about 0.03 mg; about 0.04 mg; about 0.05 mg; about 0.06 mg; about 0.07 mg; about 0.08 mg; about 0.09 mg; about 0.1 mg; about 0.2 mg; about 0.3 mg; about 0.4 mg; about 0.5 mg; about 0.6 mg; about 0.7 mg; about 0.8 mg; about 0.9 mg; about 1 mg; about 1.5 mg; about 2 mg; about 2.5 mg; about 3 mg; about 3.5 mg; about 4 mg; about 4.5 mg; about 5 mg; about 5.5 mg; about 6 mg; about 6.5 mg; about 7 mg; about 7.5 mg; about 8 mg; about 8.5 mg; about 9 mg; about 9.5 mg; about 10 mg; about 10.5 mg; about 11 mg; about 11.5 mg; about 12 mg; about 12.5 mg; about 13 mg; about 13.5 mg; about 14 mg; about 14.5 mg; about 15 mg; about 15.5 mg; about 16 mg; about 16.5 mg; about 17 mg; about 17.5 mg; about 18 mg; about 18.5 mg; about 19 mg; about 19.5 mg; about 20 mg; about 20.5 mg; about 21 mg; about 21.5 mg; about 22 mg; about 22.5 mg; about 23 mg; about 23.5 mg; about 24 mg; about 24.5 mg; about 25 mg; about 25.5 mg; about 26 mg; about 26.5 mg; about 27 mg; about 27.5 mg; about 28 mg; about 28.5 mg; about 29 mg; about 29.5 mg; about 30 mg; about 30.5 mg; about 31 mg; about 31.5 mg; about 32 mg; about 32.5 mg; about 33 mg; about 33.5 mg; about 34 mg; about 34.5 mg; about 35 mg; about 35.5 mg; about 36 mg; about 36.5 mg; about 37 mg; about 37.5 mg; about 38 mg; about 38.5 mg; about 39 mg; about 39.5 mg; about 40 mg; about 40.5 mg; about 41 mg; about 41.5 mg; about 42 mg; about 42.5 mg; about 43 mg; about 43.5 mg; about 44 mg; about 44.5 mg; about 45 mg; about 45.5 mg; about 46 mg; about 46.5 mg; about 47 mg; about 47.5 mg; about 48 mg; about 48.5 mg; about 49 mg; about 49.5 mg; about 50 mg; about 50.5 mg; about 51 mg; about 51.5 mg; about 52 mg; about 52.5 mg; about 53 mg; about 53.5 mg; about 54 mg; about 54.5 mg; about 55 mg; about 55.5 mg; about 56 mg; about 56.5 mg; about 57 mg; about 57.5 mg; about 58 mg; about 58.5 mg; about 59 mg; about 59.5 mg; about 60 mg; about 60.5 mg; about 61 mg; about 61.5 mg; about 62 mg; about 62.5 mg; about 63 mg; about 63.5 mg; about 64 mg; about 64.5 mg; about 65 mg; about 65.5 mg; about 66 mg; about 66.5 mg; about 67 mg; about 67.5 mg; about 68 mg; about 68.5 mg; about 69 mg; about 69.5 mg; about 70 mg; about 70.5 mg; about 71 mg; about 71.5 mg; about 72 mg; about 72.5 mg; about 73 mg; about 73.5 mg; about 74 mg; about 74.5 mg; about 75 mg; about 75.5 mg; about 76 mg; about 76.5 mg; about 77 mg; about 77.5 mg; about 78 mg; about 78.5 mg; about 79 mg; about 79.5 mg; about 80 mg; about 80.5 mg; about 81 mg; about 81.5 mg; about 82 mg; about 82.5 mg; about 83 mg; about 83.5 mg; about 84 mg; about 84.5 mg; about 85 mg; about 85.5 mg; about 86 mg; about 86.5 mg; about 87 mg; about 87.5 mg; about 88 mg; about 88.5 mg; about 89 mg; about 89.5 mg; about 90 mg; about 90.5 mg; about 91 mg; about 91.5 mg; about 92 mg; about 92.5 mg; about 93 mg; about 93.5 mg; about 94 mg; about 94.5 mg; about 95 mg; about 95.5 mg; about 96 mg; about 96.5 mg; about 97 mg; about 97.5 mg; about 98 mg; about 98.5 mg; about 99 mg; about 99.5 mg; or about 100 mg. Recitation of each of these discrete values is understood to include ranges between each value.

In some embodiments, the initial autophagy inhibiting agent release rate can be between about 0.01 mcg/day and about 50 mcg/day. For example, initial autophagy inhibiting agent release rate can be about 0.01 mcg/day; about 0.02 mcg/day; about 0.03 mcg/day; about 0.04 mcg/day; about 0.05 mcg/day; about 0.06 mcg/day; about 0.07 mcg/day; about 0.08 mcg/day; about 0.09 mcg/day; about 0.1 mcg/day; about 0.2 mcg/day; about 0.3 mcg/day; about 0.4 mcg/day; about 0.5 mcg/day; about 0.6 mcg/day; about 0.7 mcg/day; about 0.8 mcg/day; about 0.9 mcg/day; about 1 mcg/day; about 1.5 mcg/day; about 2 mcg/day; about 2.5 mcg/day; about 3 mcg/day; about 3.5 mcg/day; about 4 mcg/day; about 4.5 mcg/day; about 5 mcg/day; about 5.5 mcg/day; about 6 mcg/day; about 6.5 mcg/day; about 7 mcg/day; about 7.5 mcg/day; about 8 mcg/day; about 8.5 mcg/day; about 9 mcg/day; about 9.5 mcg/day; about 10 mcg/day; about 10.5 mcg/day; about 11 mcg/day; about 11.5 mcg/day; about 12 mcg/day; about 12.5 mcg/day; about 13 mcg/day; about 13.5 mcg/day; about 14 mcg/day; about 14.5 mcg/day; about 15 mcg/day; about 15.5 mcg/day; about 16 mcg/day; about 16.5 mcg/day; about 17 mcg/day; about 17.5 mcg/day; about 18 mcg/day; about 18.5 mcg/day; about 19 mcg/day; about 19.5 mcg/day; about 20 mcg/day; about 20.5 mcg/day; about 21 mcg/day; about 21.5 mcg/day; about 22 mcg/day; about 22.5 mcg/day; about 23 mcg/day; about 23.5 mcg/day; about 24 mcg/day; about 24.5 mcg/day; about 25 mcg/day; about 25.5 mcg/day; about 26 mcg/day; about 26.5 mcg/day; about 27 mcg/day; about 27.5 mcg/day; about 28 mcg/day; about 28.5 mcg/day; about 29 mcg/day; about 29.5 mcg/day; about 30 mcg/day; about 30.5 mcg/day; about 31 mcg/day; about 31.5 mcg/day; about 32 mcg/day; about 32.5 mcg/day; about 33 mcg/day; about 33.5 mcg/day; about 34 mcg/day; about 34.5 mcg/day; about 35 mcg/day; about 35.5 mcg/day; about 36 mcg/day; about 36.5 mcg/day; about 37 mcg/day; about 37.5 mcg/day; about 38 mcg/day; about 38.5 mcg/day; about 39 mcg/day; about 39.5 mcg/day; about 40 mcg/day; about 40.5 mcg/day; about 41 mcg/day; about 41.5 mcg/day; about 42 mcg/day; about 42.5 mcg/day; about 43 mcg/day; about 43.5 mcg/day; about 44 mcg/day; about 44.5 mcg/day; about 45 mcg/day; about 45.5 mcg/day; about 46 mcg/day; about 46.5 mcg/day; about 47 mcg/day; about 47.5 mcg/day; about 48 mcg/day; about 48.5 mcg/day; about 49 mcg/day; about 49.5 mcg/day; about 50 mcg/day; about 50.5 mcg/day; about 51 mcg/day; about 51.5 mcg/day; about 52 mcg/day; about 52.5 mcg/day; about 53 mcg/day; about 53.5 mcg/day; about 54 mcg/day; about 54.5 mcg/day; about 55 mcg/day; about 55.5 mcg/day; about 56 mcg/day; about 56.5 mcg/day; about 57 mcg/day; about 57.5 mcg/day; about 58 mcg/day; about 58.5 mcg/day; about 59 mcg/day; about 59.5 mcg/day; about 60 mcg/day; about 60.5 mcg/day; about 61 mcg/day; about 61.5 mcg/day; about 62 mcg/day; about 62.5 mcg/day; about 63 mcg/day; about 63.5 mcg/day; about 64 mcg/day; about 64.5 mcg/day; about 65 mcg/day; about 65.5 mcg/day; about 66 mcg/day; about 66.5 mcg/day; about 67 mcg/day; about 67.5 mcg/day; about 68 mcg/day; about 68.5 mcg/day; about 69 mcg/day; about 69.5 mcg/day; about 70 mcg/day; about 70.5 mcg/day; about 71 mcg/day; about 71.5 mcg/day; about 72 mcg/day; about 72.5 mcg/day; about 73 mcg/day; about 73.5 mcg/day; about 74 mcg/day; about 74.5 mcg/day; about 75 mcg/day; about 75.5 mcg/day; about 76 mcg/day; about 76.5 mcg/day; about 77 mcg/day; about 77.5 mcg/day; about 78 mcg/day; about 78.5 mcg/day; about 79 mcg/day; about 79.5 mcg/day; about 80 mcg/day; about 80.5 mcg/day; about 81 mcg/day; about 81.5 mcg/day; about 82 mcg/day; about 82.5 mcg/day; about 83 mcg/day; about 83.5 mcg/day; about 84 mcg/day; about 84.5 mcg/day; about 85 mcg/day; about 85.5 mcg/day; about 86 mcg/day; about 86.5 mcg/day; about 87 mcg/day; about 87.5 mcg/day; about 88 mcg/day; about 88.5 mcg/day; about 89 mcg/day; about 89.5 mcg/day; about 90 mcg/day; about 90.5 mcg/day; about 91 mcg/day; about 91.5 mcg/day; about 92 mcg/day; about 92.5 mcg/day; about 93 mcg/day; about 93.5 mcg/day; about 94 mcg/day; about 94.5 mcg/day; about 95 mcg/day; about 95.5 mcg/day; about 96 mcg/day; about 96.5 mcg/day; about 97 mcg/day; about 97.5 mcg/day; about 98 mcg/day; about 98.5 mcg/day; about 99 mcg/day; about 99.5 mcg/day; or about 100 mcg/day. Recitation of each of these discrete values is understood to include ranges between each value. Recitation of each of a range is understood to include discrete values within the range. Generally, copper IUDs primarily work by disrupting sperm motility and damaging sperm so that they are prevented from joining with an egg. Copper acts as a spermicide within the uterus, increasing levels of copper ions, prostaglandins, and white blood cells within the uterine and tubal fluids. The increased copper ions in the cervical mucus inhibit the sperm's motility and viability, preventing sperm from traveling through the cervical mucus, or destroying it as it passes through. Copper can also alter the endometrial lining, but studies show that while this alteration can prevent implantation of a fertilized egg (“blastocyst”), it cannot disrupt one that has already been implanted.

Most copper IUDs have a plastic T-shaped frame that is wound around with pure electrolytic copper wire and/or has copper collars (sleeves). The arms of the frame hold the IUD in place near the top of the uterus. The Paragard TCu 380a measures 32 mm (1.26″) horizontally (top of the T), and 36 mm (1.42″) vertically (leg of the T).

Copper IUDs have a first year failure rate ranging from 0.1 to 2.2%. In the Eurogine Gold T IUD, which is made in Spain, there is a gold core, which further prevents the copper from fragmenting or corroding. GoldringMedusa is a differently-shaped German version of the Gold T. Another form of AuCu IUD is called Goldlily which is made by the Hungarian company, Radelkis. Goldlily consists of a layer of copper wires wrapped around an original layer of gold wires, and it provides electrochemical protection in addition to ionic protection.

Silver IUDs also exist. Radelkis also makes Silverlily, which is similar to Goldlily, and GoldringMedusa is available in an AgCu version as well. Nova-T 380 contains a strengthening silver core, but does not incorporate silver ions themselves to provide electrochemical protection. Other shapes of IUD include the so-called U-shaped IUDs, such as the Load and Multiload, and the frameless IUD that holds several hollow cylindrical minuscule copper beads. It is held in place by a suture (knot) to the fundus of the uterus. It is mainly available in China, Europe, and Germany, although some clinics in Canada can provide it. A framed copper IUD called the IUB SCu300 coils during deployment to form a three dimensional spherical shape and is based on a nickel titanium shape memory alloy core.

In addition to copper, noble metal and progestogen IUDs, patients in China can get copper IUDs with indomethacin. This non-hormonal compound reduces the severity of menstrual bleeding, and these coils are popular.

Advantages of the copper IUD include its ability to provide emergency contraception up to five days after unprotected sex. It is the most effective form of emergency contraception available. It works by preventing fertilization or implantation; however does not affect already implanted embryos. [It contains no hormones, so it can be used while breastfeeding, and fertility returns quickly after removal. Copper IUDs are also available in a wider range of sizes and shapes than hormonal IUDs.

The autophagy inhibiting agent can be incorporated into the framework of a hormonal IUD (e.g., substituting the hormone with the autophagy inhibiting agent). Hormonal IUDs (brand names Mirena, Skyla, and Liletta; referred to as intrauterine systems in the UK) work by releasing a small amount of levonorgestrel, a progestin. The primary mechanism of action is making the inside of the uterus fatal to sperm. They can also thin the endometrial lining and potentially impair implantation but this is not their usual function. Because they thin the endometrial lining, they reduce or even prevent menstrual bleeding, and can be used to treat menorrhagia (heavy menses), once pathologic causes of menorrhagia (such as uterine polyps) have been ruled out.

The progestin released by hormonal IUDs primarily acts locally; use of Mirena results in much lower systemic progestin levels than other very-low-dose progestogen only contraceptives.

Mirena is approved for use up to five years in the US, though studies support its efficacy for up to seven years. Skyla uses the same mechanism of action, but is smaller and releases a lower dose of levonorgestrel, and is only approved for up to three years. Liletta is more similar to Mirena in both shape and dose of levonorgestrel released; it has currently been approved for usage up to three years.

The autophagy inhibiting agent can be incorporated into an inert IUD. Inert IUDs are IUDs with no bioactive components; they are made of inert materials like stainless steel (such as the stainless steel ring, or SSR, a flexible ring of steel coils that can deform to be pushed through the cervix) or plastic (such as the Lippes Loop, which can be inserted through the cervix in a cannula and takes a trapezoidal shape within the uterus). They are less effective than copper or hormonal IUDs, with a side effect profile similar to copper IUDs. Their primary mechanism of action is inducing a local foreign body reaction, which makes the uterine environment hostile both to sperm and to implantation of an embryo. They may have higher rates of preventing pregnancy after fertilization, instead of before fertilization, compared to copper or hormonal IUDs.

Inert IUDs are not yet approved for use by the healthcare authorities in the United States, UK, or Canada. In China, where IUDs are the most common form of contraception, copper IUD production replaced inert IUD production in 1993. However, as of 2008, the most common IUD used by immigrants presenting to Canadian clinics for removal of IUDs placed in China was still the SSR. Because the SSR has no string for removal, it can present a challenge to healthcare providers unfamiliar with IUD types not available in their region.

The autophagy inhibiting agent can be incorporated into a frameless IUD. Frameless IUDs, which are available outside of the US, currently contain either copper or levonorgestrel that has been attached to a non-resorbable filament. The GyneFix 330 is made up of copper cylinders threaded onto a polypropylene suture instead of the plastic frame common to other IUDs. The FibroPlant is a frameless levonorgestrel-releasing IUD consisting of a nonresorbable thread attached to a fibrous delivery system that releases 14 or 20 mcg of levonorgestrel per day. These devices are anchored to the endometrium using an insertion technique that requires additional training, as the technique is different from the other types of IUDs. The devices manufactured prior to 1996 were associated with higher expulsion rates during the first year of use; subsequent modification to the introducer system that anchors the device to the myometrium appears to have overcome this problem, although data are sparse. Advantages of these systems include small size, high efficacy, and high tolerability. They are as effective as conventional IUDs and may be more adaptable to variations in the shape of the uterine cavity.

Current methods of IUD use to prevent pregnancy have adverse effects. Many of these adverse effects can be overcome with the use of the presently disclosed non-hormonal, non-metal-based pharmaceutical, such as the autophagy inhibiting agent. IUDs with progestogen confer an increased risk of ovarian cysts, and IUDs with copper confer an increased risk of heavier periods.

Generally, current IUDs primarily work by preventing fertilization. The progestogen released from the hormonal IUDs may prevent ovulation from occurring but only partially. The hormone also thickens the cervical mucus so that sperm cannot reach the fallopian tubes. Copper IUDs contain no hormones, but the copper ions in the cervical mucus are toxic to sperm. They also cause the uterus and fallopian tubes to produce a fluid that contains white blood cells, copper ions, enzymes, and prostaglandins, a combination that is also toxic to sperm. The very high effectiveness of copper-releasing IUDs as emergency contraceptives implies they may also act by preventing implantation of the blastocyst. In non-emergency use, prevention of implantation is at most an exceptional method of action, not a typical mechanism of action.

The IUS or IUD (IUS and IUD can be used interchangeably, herein) as described herein can be based on a T-shaped polymer frame (e.g., polyethylene), can measure any size suitable for insertion into the uterus, such as about 32 mm by 32 mm or about 28 mm by 30 mm, with a collar containing a therapeutically effective amount of an autophagy inhibiting agent dispersed in a polymer (e.g., polydimethylsiloxane) attached to a vertical stem. The diameter of the insertion tube for the IUS can be about 4.4 mm or about 3.8. The IUS can initially releases approximately a daily therapeutic dose over the course of about three, about five years, or more after insertion.

The IUD can comprise a silver ring to distinguish it on ultrasound and barium in the frame to make it detectable by radiograph.

A white or clear polymer (e.g., polyethylene monofilament) string can be knotted through the base of the IUD.

The IUD can comprise a ball (e.g., about 3 mm) at the base of the stem to decrease the risk of cervical perforation. A white or clear polymer (e.g., polyethylene monofilament) string can be knotted through this ball.

The autophagy inhibition effect of autophagy inhibitor-releasing IUDs can be primarily at the level of the endometrium. However, the level endometrial concentration from the IUD preferably does not result in a high plasma concentration; the absolute plasma autophagy inhibiting agent levels can be much lower.

The present disclosure also provides for a manufacturing process for a delivery system comprising: injection molding a body construction; and optionally, injection molding of a capsule onto the body construction wherein, the body construction or the capsule comprises an autophagy inhibiting agent.

The present disclosure also provides for a process wherein the capsule has at least a first end and a second end; the body construction has at least two locking parts, each locking part having at least a first end and a second end; the first end of each locking part having a surface adapted to face and cover one of the at least first and second ends of the capsule; the diameter of at least one of the locking parts varies along its length between the first end and the second end and the capsule is mounted between the at least two locking parts; the locking parts have the shape of a truncated cone; and/or the capsule comprises an autophagy inhibiting agent and a biocompatible polymer.

The present disclosure also provides for a process wherein the body construction comprises at least one capsule comprising the autophagy inhibiting agent; or one or more body parts.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of inhibiting autophagy in a subject at risk for becoming pregnant in need administration of a therapeutically effective amount of an autophagy inhibiting agent, so as to inhibit autophagy; halt, impair, or inhibit decidualization; inhibit or prevent pregnancy; inhibit or prevent conception; disrupt ovulation; inhibit or prevent ovulation; reduce fertility or fecundity; reduce corpra lutea; or disrupt uterine receptivity.

Methods described herein are generally performed on a subject in need thereof. A subject in need of the therapeutic methods described herein can be a subject at risk for becoming pregnant. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of an autophagy inhibiting agent is, for example, that amount that would cause the desired therapeutic effect in a subject while minimizing undesired side effects. In various embodiments, an effective amount of an autophagy inhibiting agent described herein can substantially inhibit autophagy; halt, impair, or inhibit decidualization; inhibit or prevent pregnancy; inhibit or prevent conception; disrupt ovulation; inhibit or prevent ovulation; reduce fertility or fecundity; reduce corpra lutea; or disrupt uterine receptivity.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an autophagy inhibiting agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit autophagy; halt, impair, or inhibit decidualization; inhibit or prevent pregnancy; inhibit or prevent conception; disrupt ovulation; inhibit or prevent ovulation; reduce fertility or fecundity; reduce corpra lutea; or disrupt uterine receptivity.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an autophagy inhibiting agent can occur as a single event, for example in an intrauterine device or implant.

Treatment in accord with the methods described herein can be performed prior to, concurrent with, or after conventional treatment modalities for pregnancy prevention.

Administration

Agents and compositions described herein can be administered according to methods described herein in a variety of means known to the art. The agents and composition can be used therapeutically.

As discussed above, administration can be parenteral, pulmonary, oral, topical, intradermal, intramuscular, intraperitoneal, intravenous, subcutaneous, intranasal, epidural, ophthalmic, buccal, or rectal administration. But the preferred embodiment is an intrauterine device or system if used for localized non-hormonal contraception.

Agents and compositions described herein can be administered in a variety of methods well known in the arts. Administration can include, for example, methods involving intrauterine devices or systems (IUS/IUD), oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an IUS or IUD. Typically, using such a system, an agent or composition can be administered in combination with a biodegradable or biocompatible polymeric implant that releases the agent over a controlled period of time at a selected site. Examples of polymeric materials include polyanhydrides, polyorthoesters, polyglycolic acid, polylactic acid, polyethylene, vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to an intrauterine system comprising an autophagy inhibiting agent or a delivery or insertion device. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Autophagic Inhibition: A Contraceptive Target

The following example describes autophagy as a pathway that can be exploited for contraception or to prevent pregnancy.

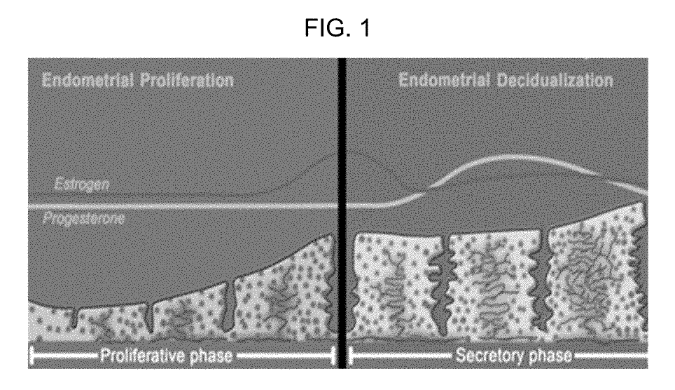

This example shows that autophagy is a normal and required process for proper endometrial decidualization and ovulation (see e.g., FIG. 1).

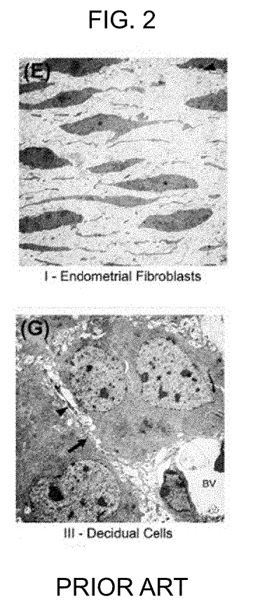

Cellular changes during decidualization can be seen in FIG. 2. Endometrial Stromal Cells (ESCs) are fibroblast-like, exhibit scant cytoplasm, elongated nuclei, and abundant Golgi and rER. Decidualized Endometrial Stromal Cells are epitheoid-like, polynucleated, exhibits an abundance of cytoplasm, ER/Golgi, free ribosomes and mitochondria which provide it secretory potential (IGFBP1 and PRL).

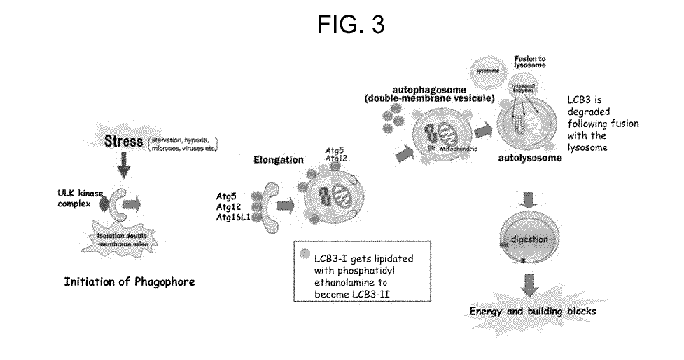

Because this requires a lot of energy, this example asks, where does this energy come from? It was hypothesized that Autophagy may be implicated (see e.g., FIG. 3).

Autophagy is a cell-protective and degradative process that recycles damaged and long-lived cellular components in response to an acute need for energy. Autophagy in endometrial stromal cells is required during the periovulatory period in order to break down macromolecules and organelles for cell energy. FIG. 4 is an illustration showing autophagy flux assay.

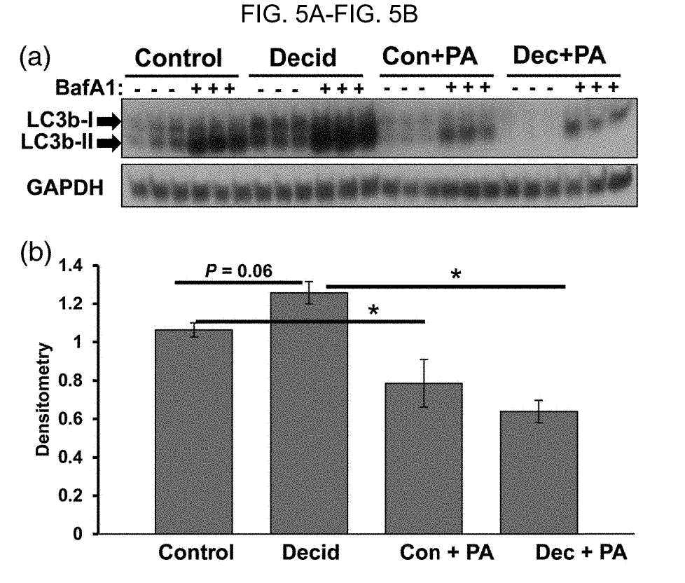

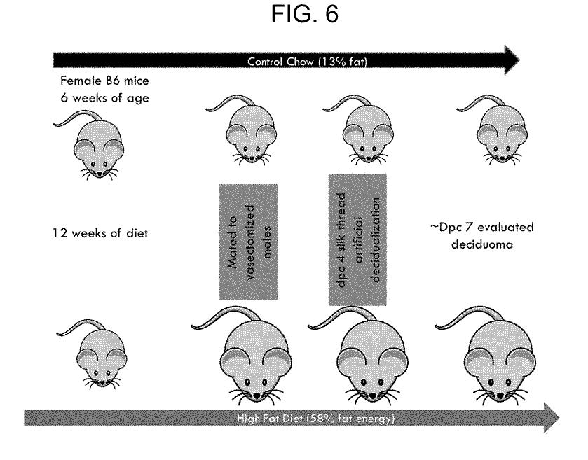

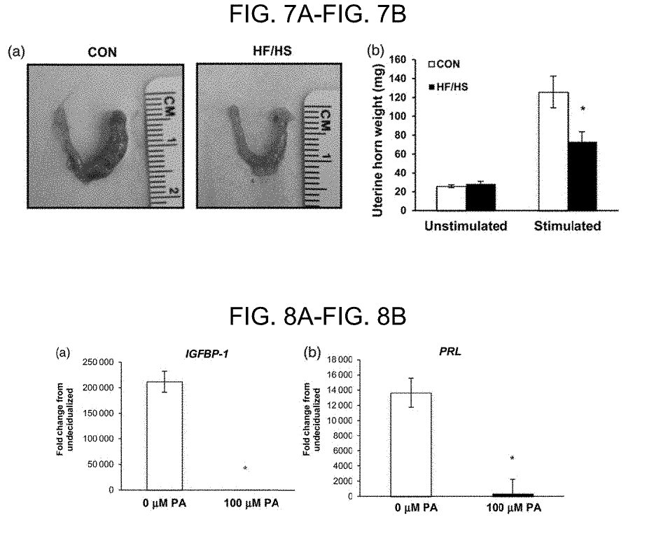

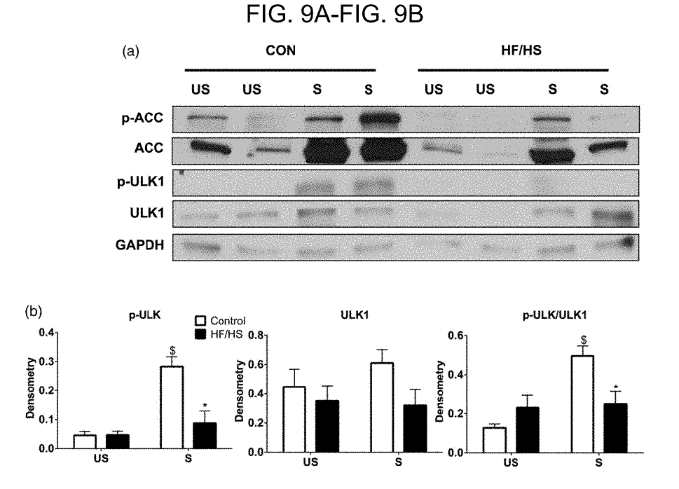

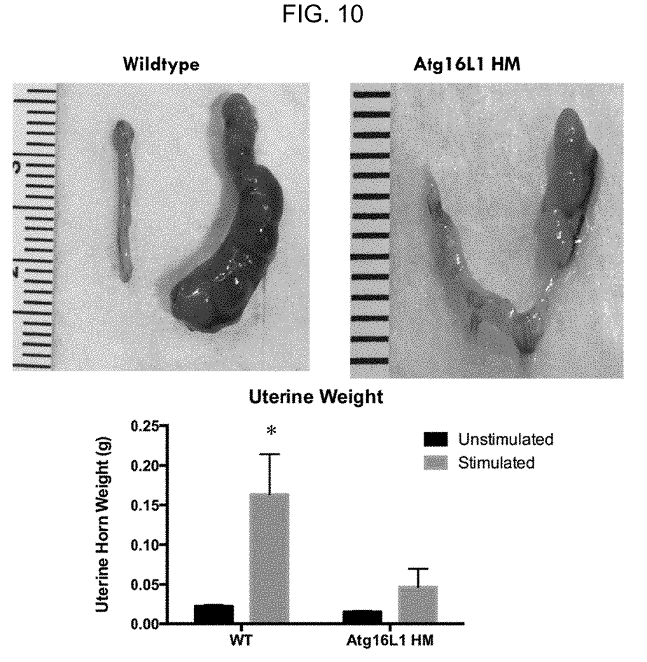

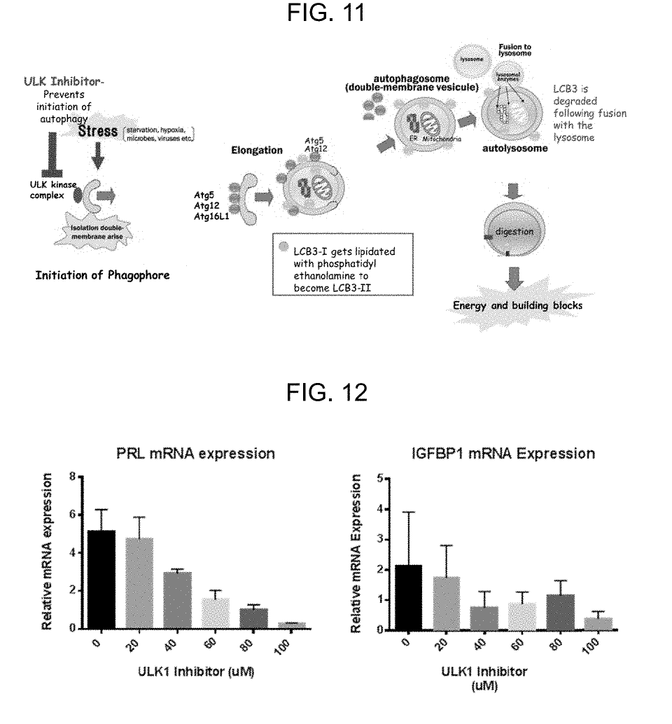

As described herein, the present disclosure shows that in obese mice, autophagy is halted, decidualization does not occur, and implantation is severely impaired. Similarly, in autophagy-deficient mice (ATG16L1 hypomorphs) litter size is smaller and fertility is impaired. Disturbances are also seen in the regression of the corpus luteum and subsequent ovulation in both models as well. It was shown that autophagy is upregulated during decidualization of human ESCs but impaired in high fat conditions (see e.g., FIG. 5). IT was shown that a high fat diet impairs artificial decidualization in mice and human endometrial stromal cells (ESCs) (see e.g., FIG. 7). It was shown that a high fat diet impairs artificial decidualization in mice and human endometrial stromal cells (ESCs) (see e.g., FIG. 8). It was shown that autophagy is up-regulated during murine decidualization but impaired with high fat diet (see e.g., FIG. 9). It was also shown that decidualization is impaired in mice with decreased autophagy (see e.g., FIG. 10).

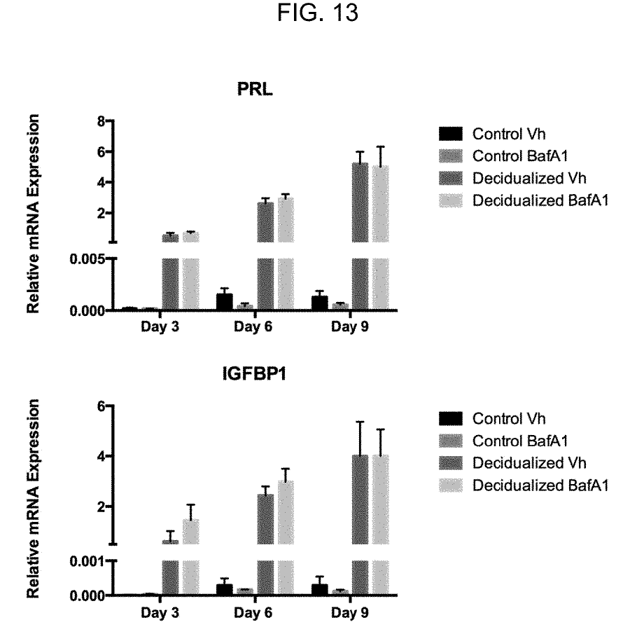

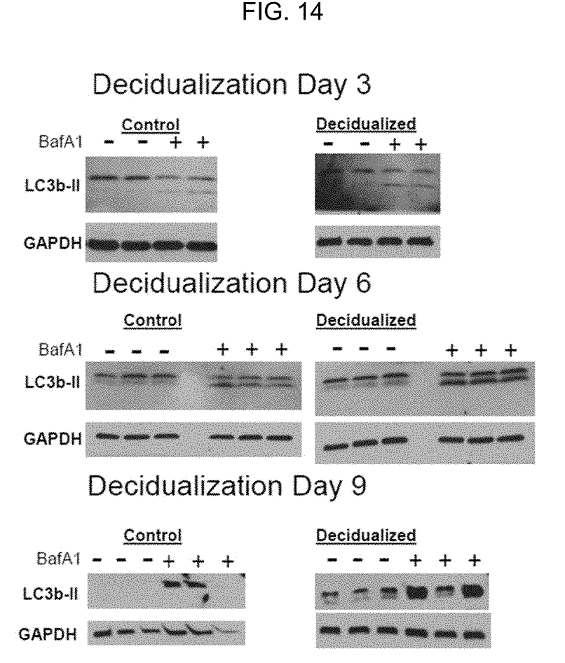

It was shown that pharmacologic inhibition of autophagy inhibits decidualization in immortalized human ESCs (see e.g., FIG. 12). I has been shown that an inhibitor of Ulk1, a serine/threonine protein kinase (presently thought to be required for the initial stages of autophagy) can impair decidualization, ovulation and thus impair fertility. This compound may be delivered by intrauterine pellets and ultimately by an IUS, in order to evaluate the efficiency of this novel target for contraception. The hypothesis is that the normal flux of the autophagic pathway in reproductive tissues is critical for successful pregnancy and thus targeting autophagy is a feasibly contraceptive target. An ULK1 inhibitor can be formulated into a LARC IUS as an efficient delivery system. As such, a composition comprising a ULK1 inhibitor can be successful in preventing pregnancy.

### Example 2: Determination of the Role of Autophagy in Decidualization

This example shows the delivery of a highly selective ULK inhibitor compound (SBI-0206965) by intrauterine pellets in the mice and ultimately by an IUS in non-human primates, in order to test the efficiency of this novel non-hormonal target for contraception. It is believed that the normal flux of the autophagic pathway in reproductive tissues is critical for successful pregnancy and thus targeting autophagy is a feasible contraceptive target.

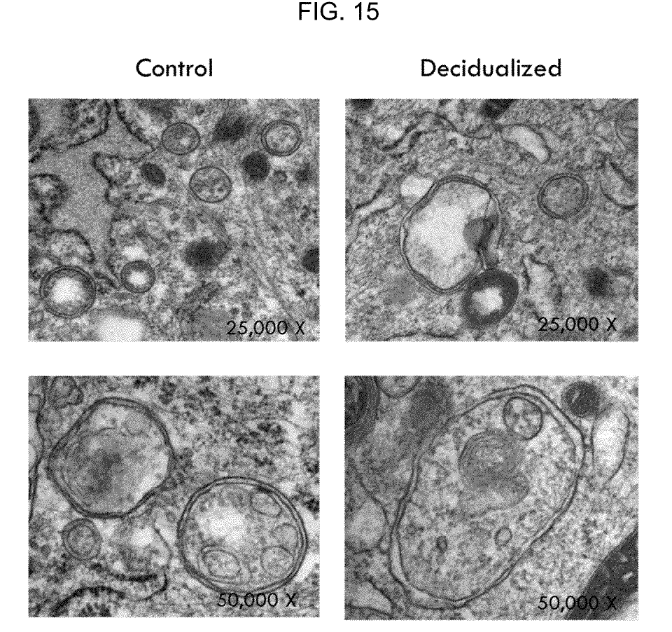

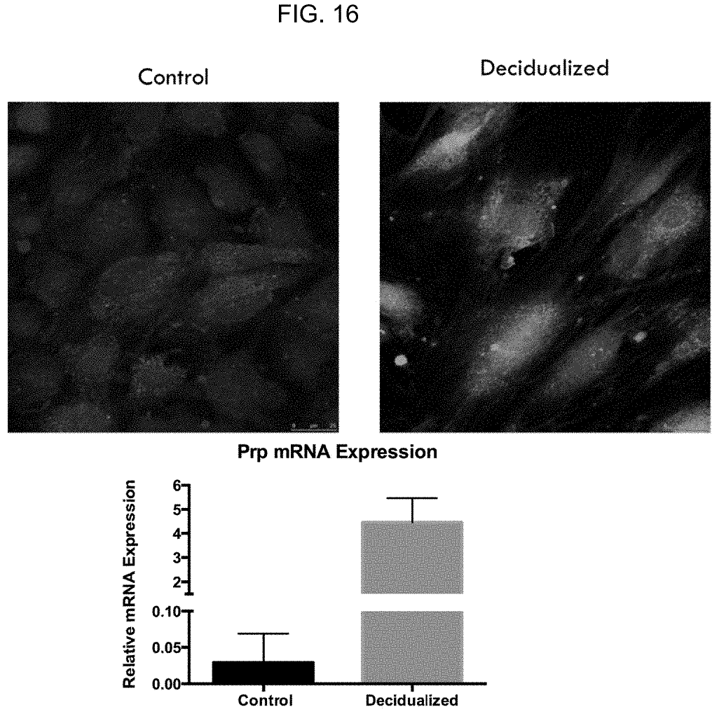

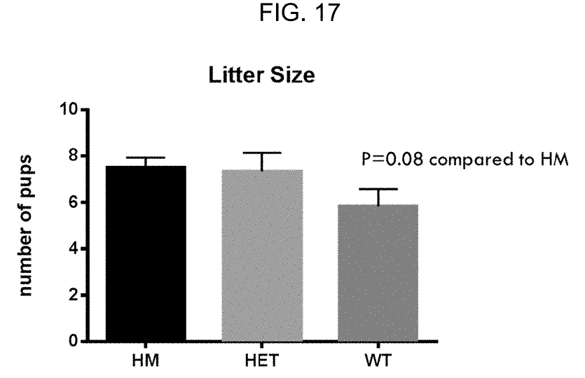

It was shown that during in vitro decidualization, the decidualization markers PRL and IGFBP1 increase in immortalized human ESCs (see e.g., FIG. 13) and autophagy is increased during in-vitro decidualization of immortalized human ESCs (see e.g., FIG. 14). It was also shown that autophagosomes are larger in decidualized immortalized human ESCs (see e.g., FIG. 15). It was shown that autophagy is also increased in ESCs cultured from LC3-GFP mice (see e.g., FIG. 16).

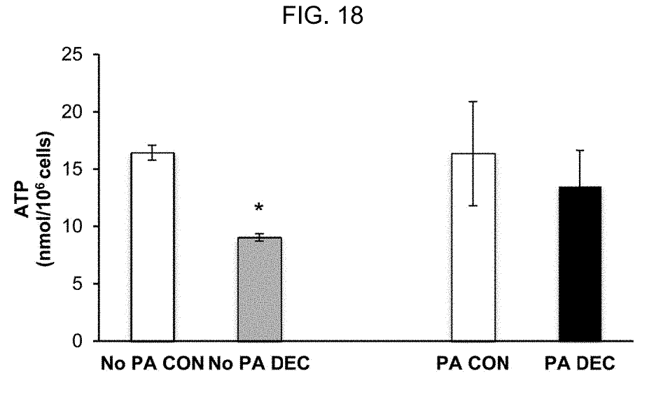

Experiments showed ATP levels decreased during decidualization but not under high fat conditions (see e.g., FIG. 18)

### Example 3: Autophagy as a Contraceptive Target

This example shows autophagy can be used as a contraceptive target.

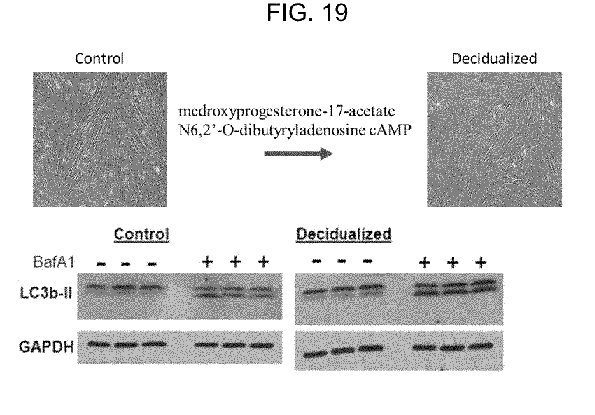

Autophagy was shown to increase during decidualization of immortalized human (hESC-t) (see e.g., FIG. 19). Autophagosomes of decidualizing hESC-t cells were shown to contain more cellular cargo (see e.g., FIG. 20).

It was shown that decidualization is impaired in Atg16L1 mice with impaired autophagy (see e.g., FIG. 22).

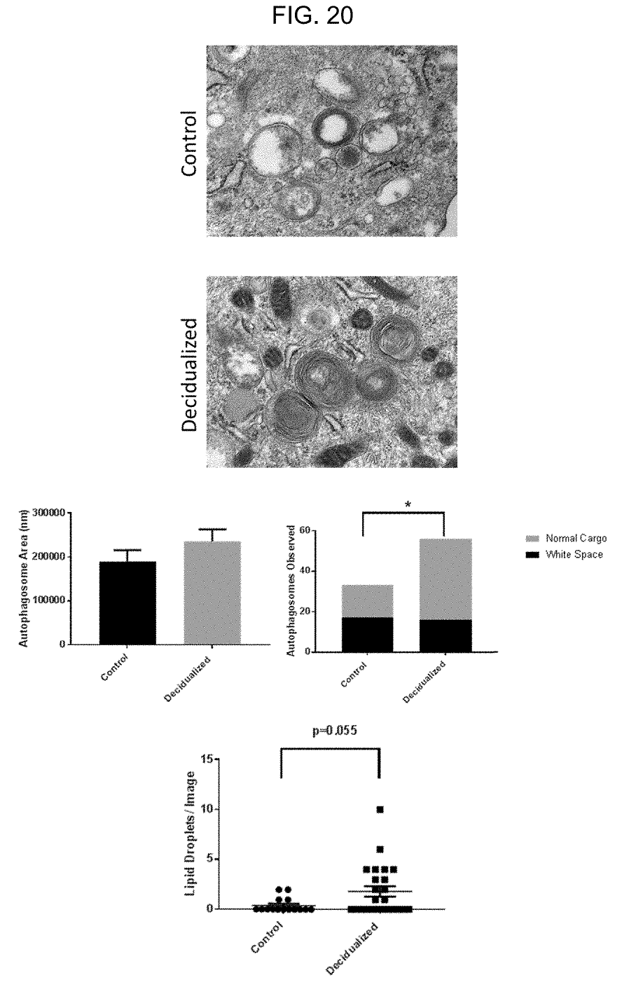

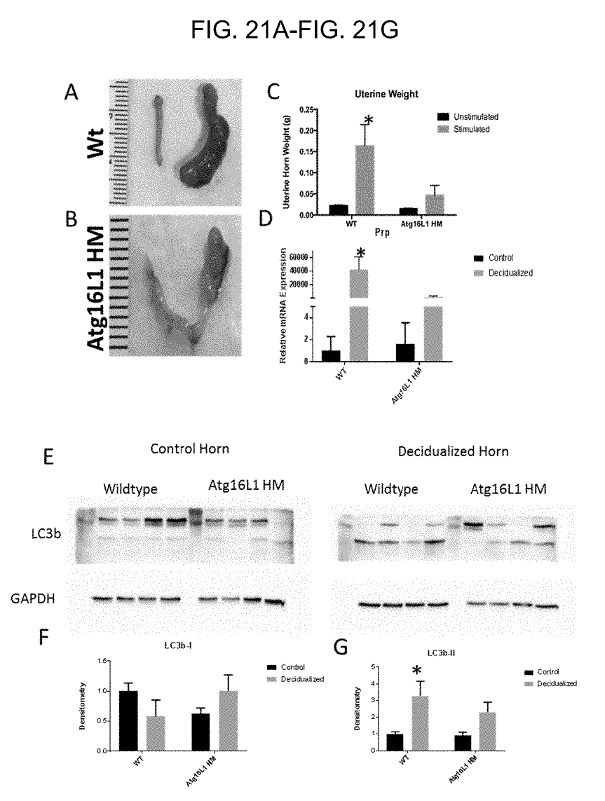

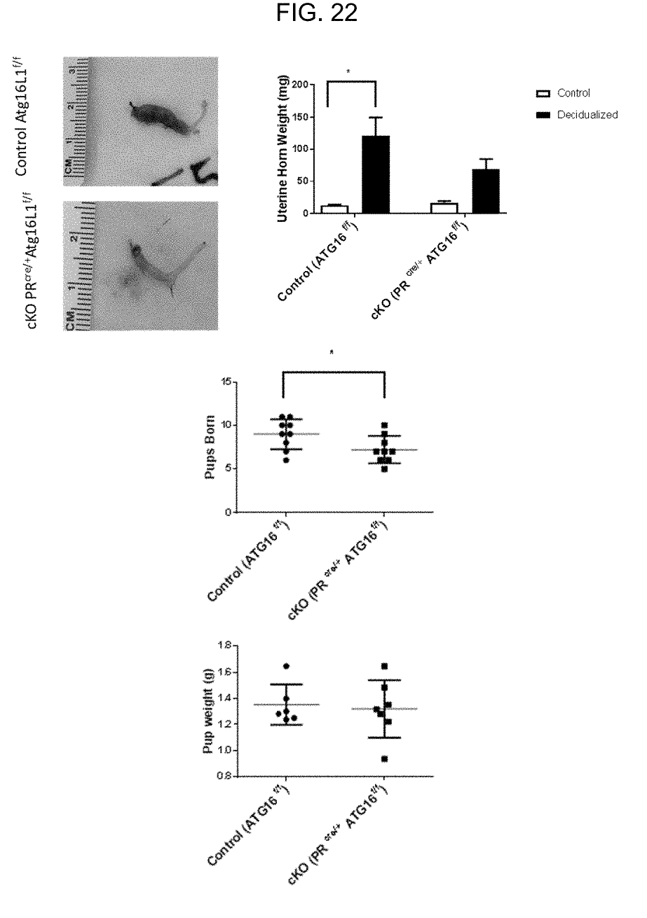

A genetic model of impaired autophagy (ATG16L1 hypomorph) demonstrated poor decidualization. Decidualization was shown to be impaired in Atg16L1 mice with impaired autophagy (see e.g., FIG. 21).

The genetic KnockOut of ATG16L1 also demonstrates decreased litter size. Uterine specific knock out of Atg16L1 was shown to impair artificial decidualization and fecundity (see e.g., FIG. 22).

### Example 4: Pharmacologic Inhibitors of Autophagy

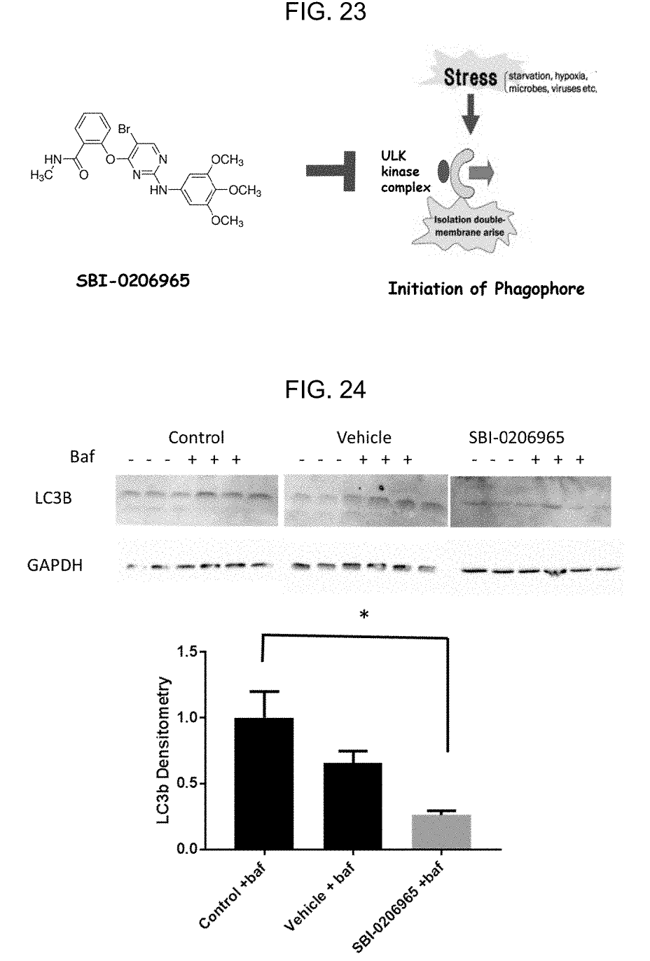

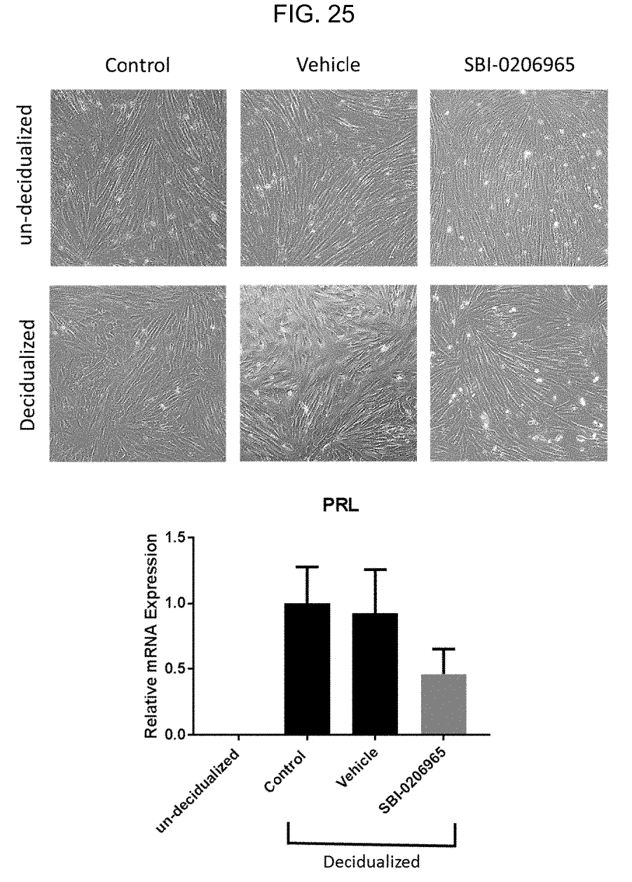

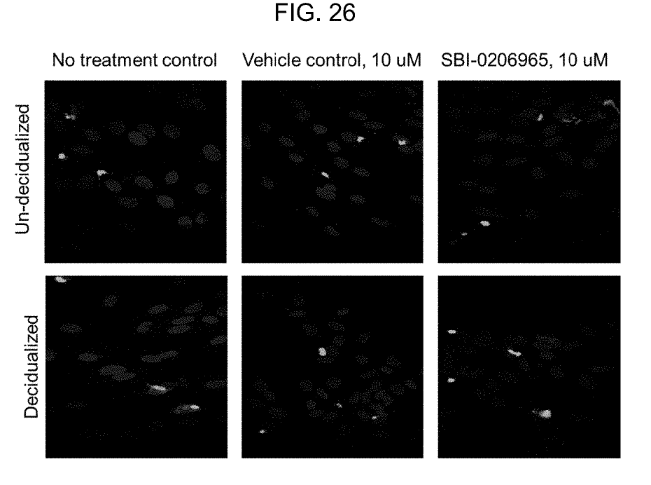

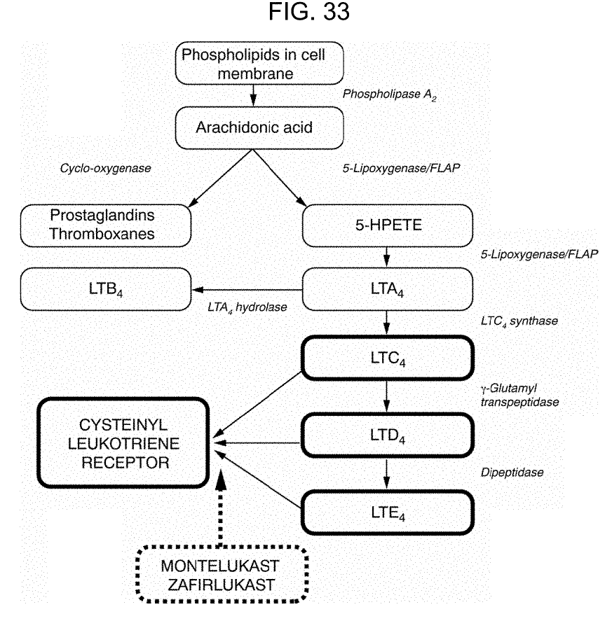

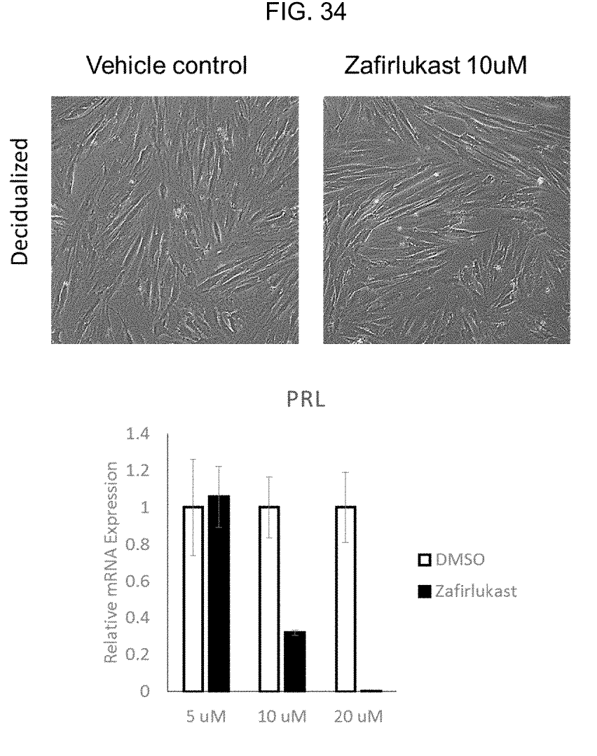

This example describes the pharmacological model and pharmacologic inhibitors of autophagy at different stages (Ulk inhibitor, see e.g., FIG. 24-FIG. 26; Zafirlukast FIG. 33-FIG. 36) inhibit decidualization both in immortalized and primary human stromal cells. These data show that these inhibitors also affect ovulation (another mechanism for contraceptive action).

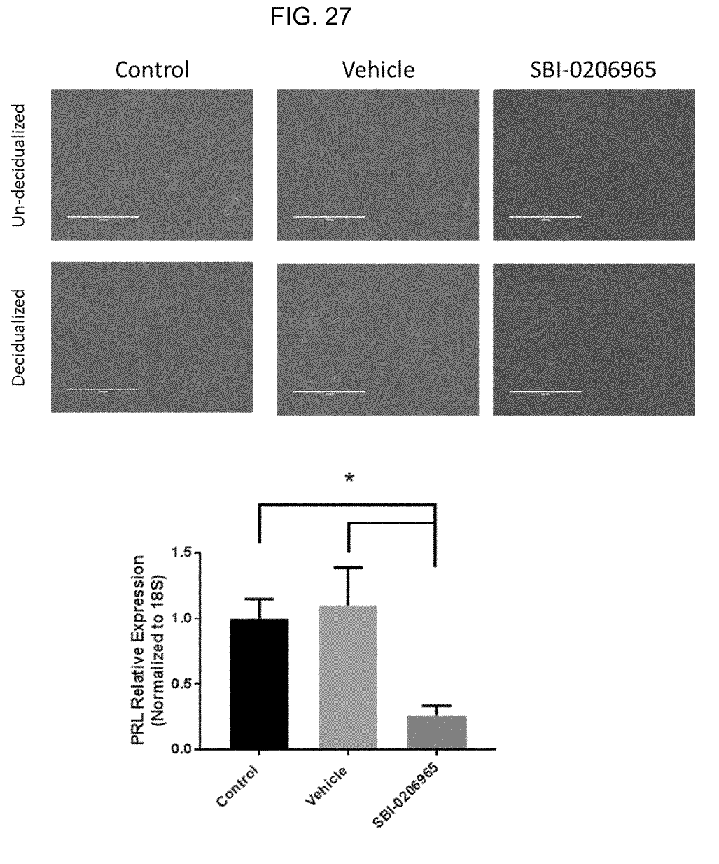

A pharmacological model was developed (see e.g., FIG. 23). Ulk inhibition was shown to decrease autophagy in hESCs-ts (see e.g., FIG. 24). Pharmacologic inhibition of autophagy in hESCs-t cells were shown to prevent morphological cellular changes indicative of decidualization (see e.g., FIG. 25). Ulk inhibition does not induce apoptosis in hESC-t cells (TUNNEL ASSAY) (see e.g., FIG. 26). Pharmacologic Inhibition of autophagy in primary hESCs-cells was shown to prevent morphological cellular changes indicative of decidualization (see e.g., FIG. 27).

Autophagy Inhibition Alters Ovulation.

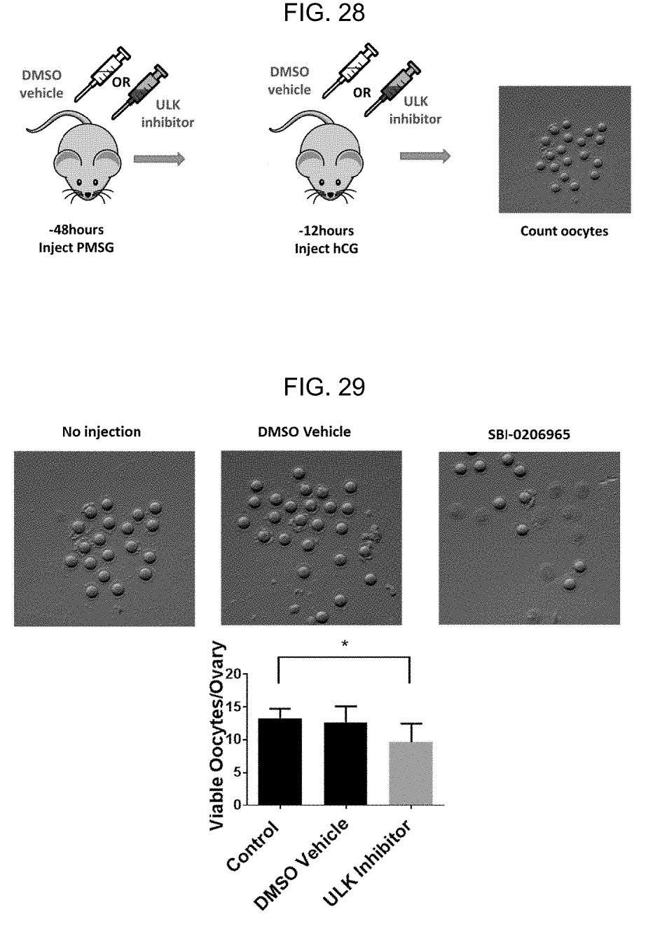

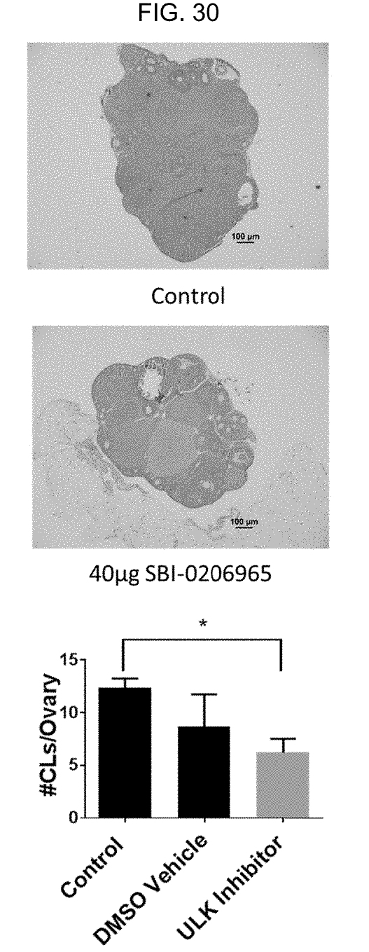

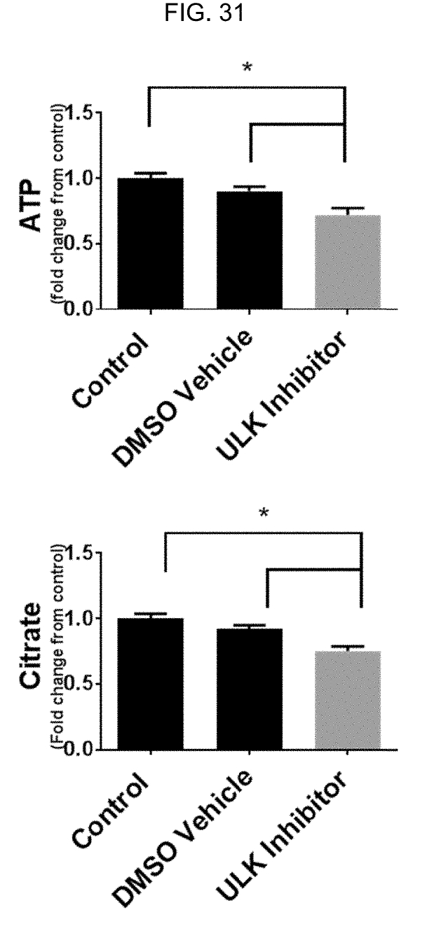

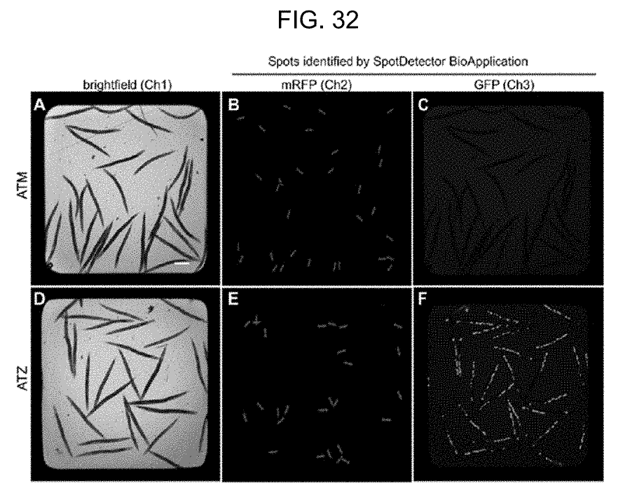

The Superovulation experimental design is illustrated in FIG. 28. An ovulation defect is shown for the autophagy inhibitor (see e.g., FIG. 29). It was shown that corpora lutea are reduced 3 days following autophagy inhibition suggesting impaired ovulation (see e.g., FIG. 30). Autophagy inhibition was shown to decrease cellular metabolites in the oocyte (see e.g., FIG. 31).

Other Inhibitors Identified from a C. elegans Drug Screen.

A C. elegans-based high-throughput screen was performed to identify novel autophagic inhibitors using the LOPAC¹²⁸⁰ library of 1280 pharmacologically active compounds from Sigma. Using this high-content screen, 27 initial hits were identified.

After validation on PubChem and examination of the worms to confirm the finding, three novel autophagy inhibitors were identified: niclosamide (PubChemID 4477), Zafirlukast (PubChemID 5717), and L(−)-vesamicol hydrochloride (PubChemID 659840).

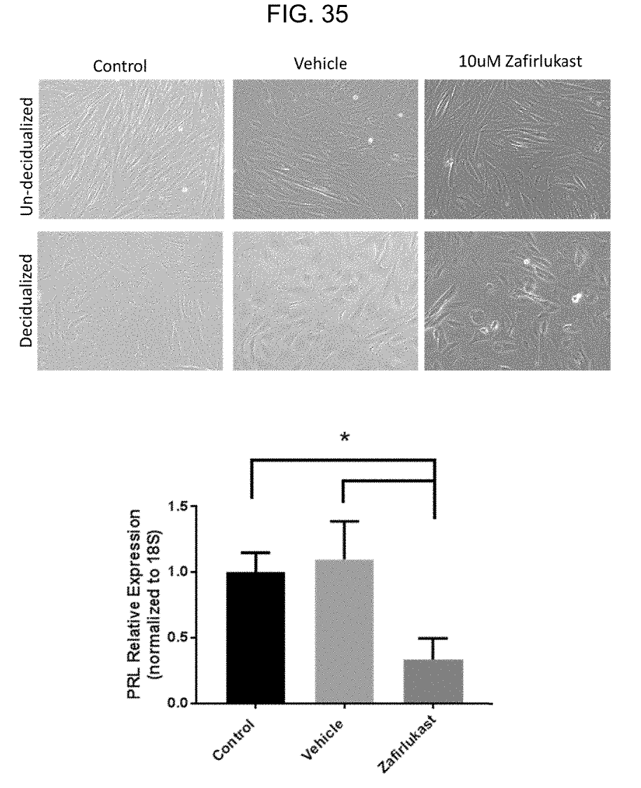

It was shown that decidualization is impaired in hESC-t cells treated with Zafirlukast (see e.g., FIG. 34). Furthermore, it was shown that decidualization is impaired in primary hESC cells treated with Zafirlukast (see e.g., FIG. 35).

What is claimed is:

1. A method of contraception comprising administration of a therapeutically effective amount of a composition comprising an autophagy inhibiting agent in a subject at risk for becoming pregnant.

2. The method of claim 1, wherein the autophagy inhibiting agent disrupts uterine receptivity, ovulation, or decidualization.

3. The method of claim 1, wherein the therapeutically effective amount of the autophagy inhibiting agent inhibits autophagy; halts, impairs, or inhibits decidualization; inhibits or prevents pregnancy; inhibit or prevent conception; disrupts ovulation; inhibits or prevents ovulation; reduces fertility or fecundity; reduces corpora lutea; or disrupts uterine receptivity.

4. The method of claim 1, wherein the subject is a fertile female with a uterus.

5. The method of claim 1, wherein the composition does not comprise a synthetic hormone.

6. The method of claim 1, wherein the subject has or is suspected of having a condition in which hormone exposure is contraindicated.

7. The method of claim 1, wherein the composition comprises one or more autophagy inhibiting agents selected from the group consisting of: niclosamide, Zafirlukast, and L(−)-vesamicol hydrochloride.

8. The method of claim 1, wherein the composition comprises one or more autophagy inhibiting agents selected from the group consisting of: SBI-0206965, niclosamide, Zafirlukast, L(−)-vesamicol, MRT68921, and LYN-1604.

9. A method of contraception comprising administering intrauterinely a composition comprising an autophagy inhibitor.

10. The method of claim 9, wherein the autophagy inhibitor is selected from the group consisting of: niclosamide, Zafirlukast, and L(−)-vesamicol hydrochloride.

11. The method of claim 9, wherein the autophagy inhibitor is selected from the group consisting of: SBI-0206965, niclosamide, Zafirlukast, L(−)-vesamicol, MRT68921, and LYN-1604.

12. The method of claim 9, wherein the composition is formulated as an intrauterine system (IUS) or intrauterine device (IUD).

13. A delivery system comprising a body construction suitable for use in an intrauterine system comprising at least one pharmaceutical composition comprising an autophagy inhibiting agent.

14. The delivery system of claim 13, wherein the body construction comprises at least one pharmaceutical composition; or the body construction comprises a biocompatible polymer.

15. The delivery system of claim 13, wherein
the body construction comprises a capsule;
the capsule has at least a first end and a second end;
the capsule comprises the at least one pharmaceutical composition comprising a pharmaceutically active agent;
the body construction has at least two locking parts, each locking part having at least a first end and a second end;
the first end of each locking part has a surface adapted to face and cover one of the at least first and second ends of the capsule;
the diameter of at least one of the locking parts varying along its length between the first end and the second end;
the capsule is mounted between the at least two locking parts; or
the locking parts have a truncated cone shape.

16. The delivery system of claim 13, wherein the body construction comprises two or more capsules containing a pharmaceutical composition.

17. The delivery system of claim 13, wherein the delivery system is an intrauterine system (IUS) or intrauterine device (IUD).

18. A method of reducing autophagy in a subject comprising administering a pharmaceutical composition comprising an autophagy inhibiting agent selected from the following: niclosamide, Zafirlukast, and L(−)-vesamicol hydrochloride to a subject in need thereof.

19. The method of claim 18, wherein the subject is at risk for becoming pregnant.

20. The method of claim 19, wherein the subject has a disease disorder or condition treatable with an autophagy inhibiting agent selected from the group consisting of metabolic conditions, obesity, diabetes, or cancer.
